(19) United States
(12) Patent Application Publication
(10) Pub. No.: US 2023/0416203 A1
(43) Pub. Date: Dec. 28, 2023

(54) POTENT AND SELECTIVE INHIBITORS OF THE CALCIUM-ACTIVATED POTASSIUM CHANNEL, KCA3.1, FOR USE AS PLATFORM THERAPEUTICS

(71) Applicants: Nanyang Technological University, Singapore (SG); Ice Bioscience Inc., Beijing (CN)

(72) Inventors: George Kanianthara CHANDY, Singapore (SG); Seow Theng ONG, Singapore (SG); Yingji LI, Beijing (CN)

(73) Assignees: Nanyang Technological University, Singapore (SG); Ice Bioscience Inc., Beijing (CN)

(21) Appl. No.: 18/036,969

(22) PCT Filed: Nov. 30, 2021
(86) PCT No.: PCT/SG2021/050734

§ 371 Date: May 15, 2023

(30) Foreign Application Priority Data
Nov. 30, 2020 (SG) 10202011877V

(51) Int. Cl. C07D 211/90; A61P 9/10; C07D 409/04

(52) U.S. Cl. CPC C07D 211/90; A61P 9/10; C07D 409/04

(57) ABSTRACT

Provided is the use of compounds of Formula I, as well as certain specific compounds, or a pharmaceutically acceptable salt, solvate or derivative thereof, in the preparation of a medicament to treat or prevent a disease condition that is associated with increased K_{Ca}3.1 or altered activity, such as stroke.

### FIELD OF INVENTION

The invention relates to the use of a compound of formula I, and certain specific compounds, and to pharmaceutically acceptable salts, solvates and derivatives thereof, in the preparation of a medicament to treat or prevent a disease condition that is associated with increased K_{Ca}3.1 or altered activity.

### BACKGROUND

The listing or discussion of a prior-published document in this specification should not necessarily be taken as an acknowledgement that the document is part of the state of the art or is common general knowledge.

K_{Ca}3.1 (KCNN4), one of 78 potassium channel genes in the human genome, encodes the intermediate-conductance calcium-activated potassium channel (S. P. Alexander et al., Br. J. Pharmacol. 2017, 174 Suppl 1, S1-S16; G. A. Gutman et al., Pharmacol. Rev. 2005, 57, 473-508; L. K. Kaczmarek et al., Pharmacol. Rev. 2017, 69, 1-11; and A. D. Wei et al., Pharmacol. Rev 2005, 57, 463-472). The channel is a complex of four K_{Ca}3.1 subunit, each attached to a calmodulin (CaM) that serves as the calcium sensor (FIG. 1A). The cryo-EM structure of the complex has been determined and binding sites of inhibitors and activators have been determined by mutagenesis (FIG. 11B). The Ca²⁺-activated K⁺ channel K_{Ca}3.1 functions as a cation counterbalancer to sustain calcium entry and calcium signaling in immune cells (lymphocytes, microglia, macrophages, mast cells), red blood cells, platelets, epithelial cells in lung and gastrointestinal tracts, endothelial cells, fibroblasts, myofibroblasts and some cancers. In these cells, external signals activate relevant cell surface receptors causing an increase in intracellular Ca²⁺, which opens K_{Ca}3.1 channels (FIG. 2).

Pharmacological and genetic studies have validated K_{Ca}3.1 as a therapeutic target. K_{Ca}3.1 inhibitors reduce vascular stenosis and atherosclerosis in rodent and pig models (D. Tharp et al., Arterioscler Thromb. Vasc. Biol. 2008, 28, 1084-1089; K. Toyama et al., J. Clin. Invest. 2008, 118, 3025-3037; R. Kohler et al., Circulation 2003, 108, 1119-1125). K_{Ca}3.1 blockade or genetic knockout of K_{Ca}3.1 ameliorates disease in rodent models of inflammatory bowel disease (L. Di et al., Proc. Natl. Acad. Sci. USA 2010, 107, 1541-1546; D. Strobaek et al., Br. J. Pharmacol. 2013, 168, 432-444; S. Ohya et al., Am. J. Physiol. Gastrointest. Liver Physiol. 2014, 306, G873-885), multiple sclerosis (E. Reich et al., Eur. J. Immunol. 2005, 35, 1027-1036), glomerulonephritis (H. Kang et al., Cell Rep. 2014, 8, 1210-1224), inflammatory arthritis (H. Kang et al., Cell Rep. 2014, 8, 1210-1224), bone resorption (H. Kang et al., Cell Rep. 2014, 8, 1210-1224), allergic rhinitis (H. Lin et al., Sci. Rep. 2015, 5, 13127; H. Lin et al., Int. Immunopharmacol. 2014, 23, 642-648), conjunctival and corneal fibrosis (H. Yang et al., Exp Eye Res 2013, 110, 76-87; G. Anumanthan et al., PLoS One 2018, 13), cardiac fibrosis (L. Zhao et al., Pflugers Arch. 2015, 467, 2275-2285; L. Wang et al., Pflugers Arch. 2016, 468, 2041-2051), lung fibrosis (D. Amrutkar Experimental Biology Meeting 2021; L. Organ et al., Am. J. Respir. Cell Mol. Biol. 2017, 56, 539-550; L. Organ et al., Am. J. Respir. Cell Mol. Biol. 2017, 56, 539-550; U. Perera et al., Can. Respir. J. 2021, 6683, 1955), diabetic renal disease (C. Huang et al., Diabetes 2013, 62, 2923-2934; C. Huang et al., PLoS One 2018, 13, e0192800; I. Grgic et al., Proc. Natl. Acad. Sci. USA 2009, 106, 14518-14523; C. Huang et al., Nephrology Dialysis Transplantation 2014, 29(2), 313-324; C. Huang et al., Sci. Rep. 2016, 6, 23884), diet-induced hepatosteatosis, non-alcoholic steatosis and liver fibrosis (L. Paka et al., World J. Gastroenterol. 2017, 23, 4181-4190; C. Freise and U. Querfeld. Pharmacol. Res. 2014, 85, 6-14), and sickle cell disease (L. De Franceschi et al., J. Clin. Invest. 1994, 93, 1670-1676; J. Stocker et al., Blood 2003, 101(6), 2412-8; K. Ataga et al., Pharmacotherapy 2006, 26, 1557-1564; K. Ataga et al., Blood 2008, 111(8), 3991-7; K. Ataga et al., Expert. Opin. Investig. Drugs 2009, 8(2), 231-9; K. Ataga et al., Br J Haematol. 2011, 153(1), 92-104; K. Ataga et al., Br J Haematol. 2021, 192(5), e129-e132). In the nervous system, K_{Ca}3.1 modulates microglial activation and inhibitors of the channel suppress microglia-mediated neuronal damage in animal models of stroke (Y. Chen et al., J. Cereb. Blood Flow Metab. 2011, 31, 2363-2374; Y. Chen et al., J. Cereb. Blood Flow Metab. 2016, 36, 2146-2161; M. Yi et al., J. Neuroinflammation 2017, 14, 203), Alzheimer's disease (M. Yi et al., Mol. Cell Neurosci. 2016, 76, 21-32; T. Wei et al., Front. Pharmacol. 2016, 7, 528; L. Jin et al., Ann. Clin. Transl. Neurol. 2019, 6, 723-738), Parkinson's disease (J. Lu et al., J. Neuroinflammation 2019, 16, 273) and traumatic brain injury (F. Mauler et al., Eur. J. Neurosci. 2004, 20, 1761-1768; K. Urbahns et al., Bioorg. Med. Chem. Lett. 2005, 15, 401-404; K. Urbahns et al., Bioorg. Med. Chem. Lett. 2003, 13, 2637-2639). K_{Ca}3.1 blockers treat lung fibrosis in rodent and sheep models, and cardiac and renal fibrosis in rodent models, via inhibition of myofibroblasts (L. Organ et al., Am. J. Respir. Cell Mol. Biol. 2017, 56, 539-550; L. Organ et al., Am. J. Respir. Cell Mol. Biol. 2017, 56, 539-550; U. Perera et al. Can. Respir. J. 2021, 6683, 1955; L. Zhao et al., Pflugers Arch. 2015, 467, 2275-2285; L. Wang et al., Pflugers Arch. 2016, 468, 2041-2051; C. Huang et al., Diabetes 2013, 62, 2923-2934; C. Huang et al., PLoS One 2018, 13, e0192800; I. Grgic et al., Proc. Natl. Acad. Sci. USA 2009, 106, 14518-14523; C. Huang et al., Nephrology Dialysis Transplantation 2014, 29(2), 313-324; C. Huang et al., Sci. Rep. 2016, 6, 23884). Shortened survival in patients with malignant glioma is associated with higher K_{Ca}3.1 expression in the tumors, and K_{Ca}3.1 blockers reduce tumor-invasiveness in rodent xenograft models (G. D'Alessandro et al., Cell Death Dis. 2013, 4, e773; A. Grimaldi et al., Cell Death Dis. 2016, 7, e2174; K. Turner et al., Glia 2014, 62, 971-981; G. D'Alessandro et al., Oncotarget 2016, 7, 30781-30796). Genetic and pharmacological studies have also validated K_{Ca}3.1 as a therapeutic target for cancer of the liver (P. Song et al., J. Cancer 2017, 8, 1568-1578), ovary (Z. Wang et al., Oncogene 2007, 26, 5107-5114), colon (N. Sassi et al., Biochim. Biophys. Acta. 2010, 1797(6-7), 1260-7; U. De Marchi et al., Cell Calcium 2009, 45(5), 509-16), rectum (H. Xu et al., BMC Cancer 2014, 10(14), 330; W. Lai et al., Med. Oncol. 2013, 30(2), 566; W. Lai et al., Oncol. Rep. 2011, 26(4), 909-17), pancreas (H. Jager et al., Mol. Pharmacol. 2004, 65(3), 630-638) and leukaemia (E. Grössinger et al., Leukemia 2014, 28, 954-958). Another potential indication is hereditary xerocytosis, an autosomal dominant congenital hemolytic anemia where the affected red blood cells (RBCs) are characterized by a cation leak of the red cell membrane accompanied by water loss, decreased potassium content, and altered hematological parameters (I. Andolfo et al., Am. J. Hematol. 2015, 90, 921-926; E. Fermo et al., Sci. Rep. 2017, 7, 1744; R. Rapetti-Mauss et al., Haematologica 2017, 102, e415-e418; R. Rapetti-Mauss et al., Haematologica 2016, 101, e431-e435; and A. Rivera et al., Am. J. Physiol. Cell Physiol. 2019, 317, C287-C302). In a subset of these patients, gain-of-function mutations of K_{Ca}3.1 cause a loss of KCl and water, leading to red blood cell shrinkage and anemia (E. Fermo et al., Sci. Rep. 2017, 7, 1744; A. Rivera et al., Am. J. Physiol. Cell Physiol. 2019, 317, C287-C302). A K_{Ca}3.1 blocker is therefore predicted to reverse these changes and improve the hematological parameters (R. Rapetti-Mauss et al., Haematologica 2016, 101, e431-e435). Pharmacological blockade of K_{Ca}3.1 has already been shown to improve haematological parameters in animal models and humans with sickle cell anemia by preventing the loss of KCl and water, and reducing RBC shrinkage (J. Stocker et al., Blood 2003, 101(6), 2412-8; K. Ataga et al., Blood 2008, 111(8), 3991-7; K. Ataga et al., Expert. Opin. Investig. Drugs 2009, 8(2), 231-9; K. Ataga et al., Br J Haematol. 2011, 153(1), 92-104; K. Ataga et al., Br J Haematol. 2021, 192(5), e129-e132; C. Brugnara et al., J. Clin. Invest. 1993, 92(1), 520-6; S. Alper et al., Blood Cells Mol. Dis. 2009, 41(1), 22-34). Other potential indications for K_{Ca}3.1 inhibitors are asthma (Z. Yu et al., Front. Pharmacol. 2017, 8, 559; L. Chachi et al., J Immunol 2013, 191, 2624-2636; J. Der Velden et al., PLoS One 2013, 8, e66886; Z H. Yu et al., Am. J. Respir. Cell. Mol. Biol. 2013, 48(6), 685-93; D. Hynes et al., Steroids 2019, 19, 151, 108459; P. Girodet et al., Am. J. Respir. Cell Mol. Biol. 2013, 48(2), 212-9; https://ichgcp.net/clinical-trials-registry/NCT00861185), allergic rhinitis (H. Lin et al., Sci. Rep. 2015, 5, 13127), secretory diarrhea (P. Rufo et al., J. Clin. Invest. 1997, 100, 3111-3120) and cystic fibrosis (A. Philp et al., Sci. Rep. 2018, 8, 9320) based on experimental studies in animal models.

However, molecular inhibitors of K_{Ca}3.1 such as TRAM-34, NS6180, 4-phenyl-4-pyrans and cyclohexadienes, have not advanced to clinical trials because of poor oral bioavailability and pharmacokinetic properties. On the other hand, although Senicapoc (ICA-17043) advanced to clinical trials and was safe, its patent has expired.

Therefore, there is a need to discover orally bioavailable, potent and selective K_{Ca}3.1 inhibitor with good pharmacokinetic properties.

### SUMMARY OF INVENTION

It has been surprisingly found that a series of phenyl-dihydropyridines displays the desired properties. Aspects and embodiments of the invention will now be discussed by reference to the following numbered clauses.

1. A compound of formula I:

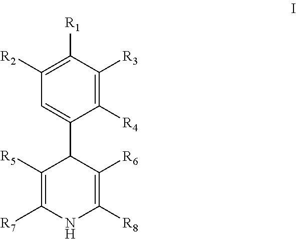

where:
R₁ is selected from H, halo, CF₃, CN or NO₂;
R₂ and R₃ are independently selected from H, halo, CH₃, CF₃, CN or NO₂;
R₄ is selected from H, halo, CN or CF₃;
R₅ and R₆ are independently selected from R₉ₐC(O)O—, R_{9b}OC(O)—, R_{9c}C(O)NR_{d}—, R₉ₑR_{9f}NC(O)—, or an alkyl ketone having from 1 to 10 carbon atoms, which carbon atoms are branched or unbranched and are unsubstituted or substituted by one of more substituents selected from halo, and NO₂;
R₇ and R₈ are independently selected from H, NR₁₀ₐR_{10b}, OR_{10c} or C₁ to C₃ alkyl which is unsubstituted or substituted by one or more substituents selected from halo, or
   one of the pair of R₅ and R₇ or R₆ and R₃, together with the carbon atoms that they are attached to, form a 4- to 14-membered ring system that is carbocyclic or heterocyclic and which is unsubstituted or substituted by one or more substituents selected from halo, ═O, —OC(O)R_{10d}, —(O)COR₁₀ₑ, and C₁ to C₃ alkyl;
R₉ₐ to R_{9f} and R₁₀ₐ to R₁₀ₑ are independently selected from H and C₁ to C₃ alkyl which is unsubstituted or substituted by one or more substituents selected from halo,
or pharmaceutically acceptable salts and/or solvates thereof.

2. The compound according to Clause 1, wherein R₁ is selected from H, F, Cl, Br, CF₃ or NO₂.

3. The compound according to Clause 1 or Clause 2, wherein R₂ and R₃ are independently selected from H, F, Cl, Br, CH₃, CF₃ or NO₂.

4. The compound according to any one of the preceding clauses, wherein R₄ is selected from H, F, Cl, Br, or CF₃.

5. The compound according to any one of the preceding clauses, wherein R₅ and R₆ are independently selected from R₉ₐC(O)O—, R_{9b}OC(O)—, or an alkyl ketone having from 1 to 10 carbon atoms, which carbon atoms are branched or unbranched and are unsubstituted or substituted by one of more substituents selected from Cl, F, and NO₂.

6. The compound according to any one of the preceding clauses, wherein R₇ and R₈ are independently selected from H or C₁ to C₃ alkyl which is unsubstituted or substituted by one or more substituents selected from F and Cl, or
one of the pair of R₅ and R₇ or R₆ and R₃, together with the carbon atoms that they are attached to, form a 4- to 10-membered ring system that is carbocyclic or heterocyclic and which is unsubstituted or substituted by one or more substituents selected from F, Cl, ═O, and C₁ to C₆ alkyl.

7. The compound according to any one of the preceding clauses, wherein R₉ₐ to R_{9f} and R₁₀ₐ to R₁₀ₑ are independently selected from H and C₁ to C₃ alkyl which is unsubstituted or substituted by one or more substituents selected from F and Cl.

8. The compound according to any one of the preceding clauses, wherein:
R₁ is selected from H, F, Cl, or CF₃;
R₂ and R₃ are independently selected from H, F, Cl, or CF₃;
R₄ is selected from H, F, Cl, or CF₃;
R₅ and R₆ are independently selected from R_{9b}OC(O)—, an alkyl ketone having from 1 to 3 carbon atoms, which carbon atoms are unsubstituted or substituted by one of more substituents selected from Cl and F;
R₇ and R₈ are independently selected from H, or methyl which is unsubstituted or substituted by one or more substituents selected from F and Cl.

9. The compound according to any one of the preceding clauses, wherein:
R₁ is selected from H, F, or Cl; and/or
R₂ is selected from CF₃ or, more particularly, H or F; and/or
R₃ is selected from H or CF₃; and/or
R₄ is H; and/or
R₅ and R₆ are independently selected from CH₃OC(O)— or propan-2-onyl (e.g. R₅ and R₆ are both CH₃OC(O)—); and/or
R₇ and R₈ are independently selected from H and CH₃.

10. The compound according to any one of the preceding clauses, wherein at least one of R₇ and R₈ is H.

11. The compound according to any one of the preceding clauses, wherein both of R₇ and R₉ is H.

12. The compound according to any one of Clause 1 to 10, wherein R₇ is H and R₈ is CH₃.

13. The compound according to any one of Clauses 1 to 10, wherein R₇ and R₈ are both CH₃.

14. The compound according to any one of Clauses 1 to 10, wherein R₇ is CH₃ and R₈ is H.

15. The compound according to any one of the preceding clauses selected from the list,

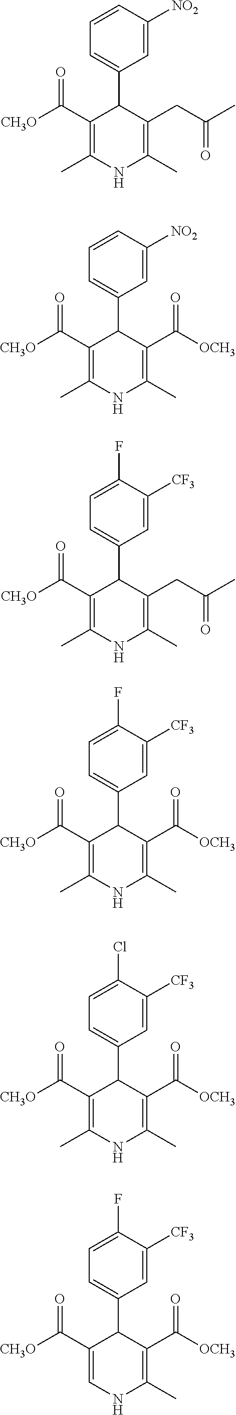

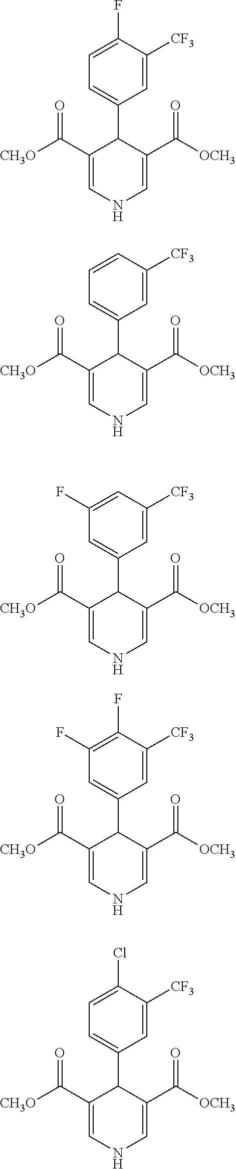

or salts and solvates thereof.

16. The compound according to any one of the preceding clauses selected from the list,

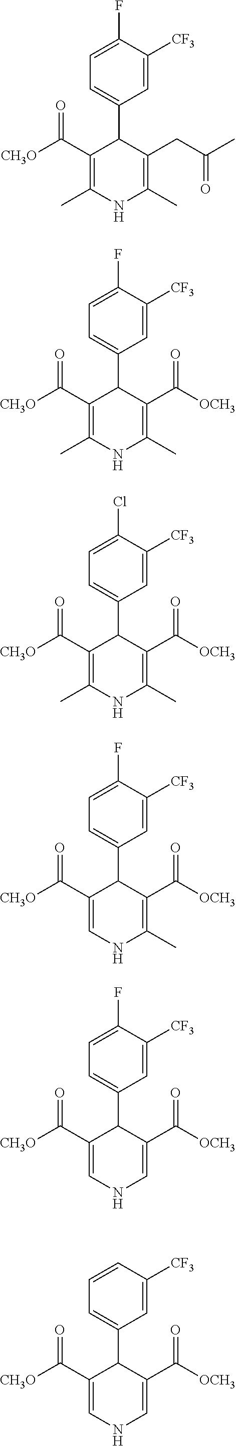

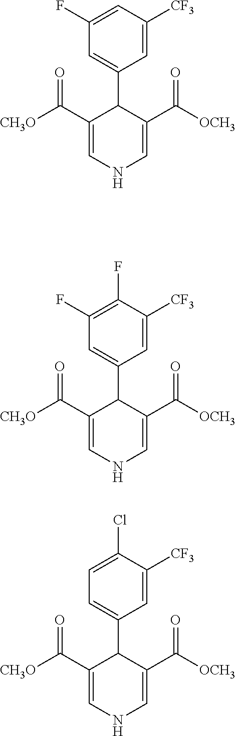

or salts and solvates thereof.

17. The compound according to any one of the preceding clauses selected from the list,

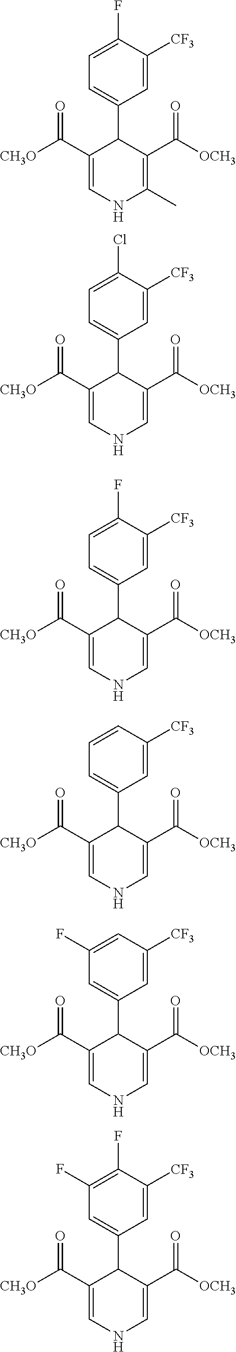

or salts and solvates thereof.

18. The compound according to any one of the preceding clauses selected from the list,

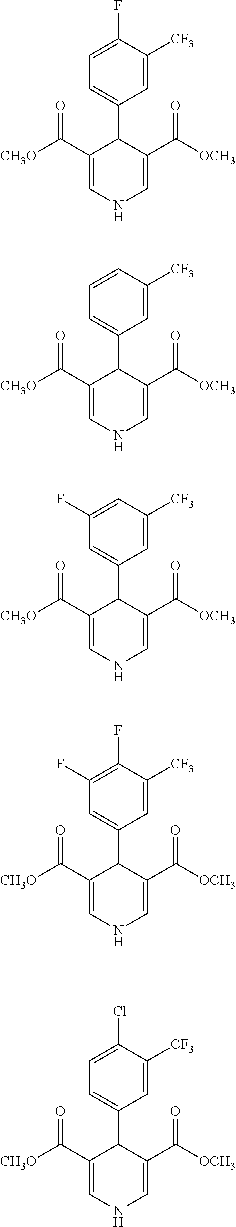

or salts and solvates thereof.

19. The compound according to any one of the preceding clauses wherein the compound is

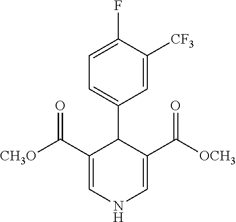

or salts and solvates thereof.

20. A pharmaceutical composition comprising a compound of formula I, or salts and solvates thereof, as described in any one of Clauses 1 to 19 in combination with one or more of a pharmaceutically acceptable carrier, adjuvant, or vehicle.

21. A compound of formula I, or salts and solvates thereof, as described in any one of Clauses 1 to 19 for use in medicine.

22. A compound of formula I, or salts and solvates thereof, as described in any one of Clauses 1 to 19 for use in the in the treatment or prevention of a disease condition that is associated with increased K_{Ca}3.1 or altered activity.

23. Use of a compound of formula I, or salts and solvates thereof, as described in any one of Clauses 1 to 19 in the preparation of a medicament for the treatment or prevention of a disease condition that is associated with increased K_{Ca}3.1 or altered activity.

24. A method of treatment or prevention of a disease condition that is associated with increased K_{Ca}3.1 or altered activity, wherein the method comprises administering an effective amount of a compound of formula, or salts and solvates thereof, as described in any one of Clauses 1 to 19 to a subject in need thereof.

25. The compound for use of Clause 19, the use of Clause 20 or the method of Clause 21, wherein the disease condition that is associated with increased K_{Ca}3.1 or altered activity is selected from one or more of leukaemia or, more particularly, inflammatory bowel diseases (IBD), fibrotic diseases (lung, liver, renal, cardiac, conjunctival, corneal), non-alcoholic fatty liver disease (NAFLD), nonalcoholic steatohepatitis (NASH), gliomas (glioblastoma), lung cancer, pancreatic cancer, hepatocellular carcinoma, ovarian cancer, colorectal cancer, cystic fibrosis, diabetic renal disease, glomerulonephritis, bone resorption, inflammatory arthritis, multiple sclerosis, atherosclerosis, restenosis following angioplasty, in-stent neo-atherosclerosis, stroke, traumatic brain injury, Alzheimer's disease, hereditary xerocytosis, sickle cell anemia, asthma, allergic rhinitis, microglial activation, nitric oxide-dependent neurodegeneration, neuro-oncological diseases and orphan red blood cell disorders.

26. The compound for use, the use or the method of Clause 25, wherein the disease condition that is associated with increased K_{Ca}3.1 or altered activity is selected from one or more of leukaemia or, more particularly, inflammatory bowel diseases (IBD), fibrotic diseases (lung, liver, renal, cardiac, conjunctival, corneal), non-alcoholic fatty liver disease (NAFLD), nonalcoholic steatohepatitis (NASH), gliomas (glioblastoma), lung cancer, pancreatic cancer, hepatocellular carcinoma, ovarian cancer, colorectal cancer, cystic fibrosis, diabetic renal disease, glomerulonephritis, bone resorption, inflammatory arthritis, multiple sclerosis, atherosclerosis, restenosis following angioplasty, in-stent neo-atherosclerosis, stroke, traumatic brain injury, Alzheimer's disease, hereditary xerocytosis, sickle cell anemia, asthma, allergic rhinitis, microglial activation and nitric oxide-dependent neurodegeneration.

27. The compound for use, the use or the method of Clause 26, wherein the disease condition is stroke.

### DRAWINGS

FIG. 1 shows (A) each K_{Ca}3.1 subunit contains six transmembrane segments with the loop between segments 5 and 6 forming the pore. The cytoplasmic C-terminus is constitutively bound to CaM (C. M. Fanger et al., J Biol Chem 1999, 274, 5746-5754). Four K_{Ca}3.1 subunits and four CaMs form the functional channel. Figure is taken from H. Wulff & N. A. Castle, Expert Rev. Clin. Pharmacol. 2010, 3, 385-396; and (B) Cryo-EM structure of the K_{Ca}3.1-CaM complex. Binding sites of blockers are shown. Peptide inhibitors bind to the channel's outer mouth. Small molecule inhibitors bind in the inner pore or in a window region in the inner pore. Small molecule activators bind to the inner surface of the K_{Ca}3.1-CaM complex. Figure is taken from B. M. Brown et al., Annu. Rev. Pharmacol. Toxicol. 2020, 60, 219-240.

FIG. 2 shows the physiological role of K_{Ca}3.1. (A) shows that K_{Ca}3.1 plays physiologically important roles in immune cells (microglia, T cells, B cells, mast cells, monocytes, macrophages), red blood cells, platelets, epithelial cells in lung and gastrointestinal tracts, endothelial cells, fibroblasts, myofibroblasts and some cancers. In these cells, external signals activate relevant cell surface receptors causing an increase in intracellular Ca²⁺, which opens K_{Ca}3.1 channels. The channel functions as a cation counterbalancer to sustain calcium signaling. Figure is taken from Wulff & N. A. Castle, Expert Rev. Clin. Pharmacol. 2010, 3, 385-396; (B) shows that many cell types can differentiate into proto-myofibroblasts. When stimulated by transforming growth factor beta (TGF-b), platelet-derived growth factor (PDGF), epidermal growth factor (EGF), fibronectin, mechanical tension etc., proto-myofibroblasts differentiate into myofibroblasts that contribute to the pro-fibrotic effects in fibrosis in many organs. Myofibroblasts up-regulate K_{Ca}3.1 expression, and their blockade suppresses myofibroblast proliferation and pro-fibrotic effects. Figure is adapted from S. W. M. van der Borne et al., Nat. Rev. Cardiol. 2010, 7, 30-37; and (C) K_{Ca}3.1 plays a key role in pro-inflammatory microglia that contribute to neuroinflammatory diseases including stroke, Alzheimer's disease, Parkinson's disease and traumatic brain injury. K_{Ca}3.1 is up-regulated as microglia differentiate into M1 pro-inflammatory cells and blockade of the channel suppresses the production of mediators of neuroinflammation including IL-1β, IL-6 and TNF-α. In contrast, when microglia differentiate into M2 anti-inflammatory cells, they down-regulate K_{Ca}3.1. Thus, K_{Ca}3.1 inhibitors preferentially target pro-inflammatory microglia. Figure is taken from S. R. Roig et al., J. Neurol. Neuromed. 2018, 3, 18-23.

FIG. 3 depicts the effect of exemplar compounds from Group 2-4 on K_{Ca}3.1 currents. Concentration-response curve of the exemplar compounds is shown.

FIG. 4 depicts the pharmacophore for potent block of K_{Ca}3.1 and >1000-fold selectivity over Ca_{V}1.2 (Group 4c).

FIG. 5 depicts the selectivity of compound 103 (tested at 3 μM) against a panel of other molecular targets (Eurofins Pharmacological P9 Diversity Panel Safety Screen).

FIG. 6 depicts the pharmacokinetic assessment of compound 103 (Group 4c) in rats following a single intravenous injection (5 mg/kg) or oral administration (50 mg/kg).

FIG. 7 depicts histopathology analysis.

FIG. 8 shows that (A) in rats subjected to 60 min of rat middle cerebral artery occlusion (MCAO) with 7 days of reperfusion, compound 103 effectively reduced infarct volume (n=28) compared to vehicle-controls, and was more effective than Edaravone, the positive control; and (B) improved neurological behavioral scores measured at 48 h (n=8) and day 7 (n=5) more effectively than Edaravone. Data represents mean±SEM, statistical significance was analysed by one-way ANOVA (A) or two-way ANOVA (B) *p<0.05, **p<0.01, ***p<0.001, ****p<0.0001.

FIG. 9 shows that compound 103 (10 mg/kg) effectively reduced Iba1⁺CD11b⁺ double positive activated microglia on day 7 in brain areas of CA1, CPu, M1 and S1, compared to MCAO or vehicle groups. Data represents mean±SEM, statistical significance was analysed by one-way ANOVA *p<0.05, n=5.

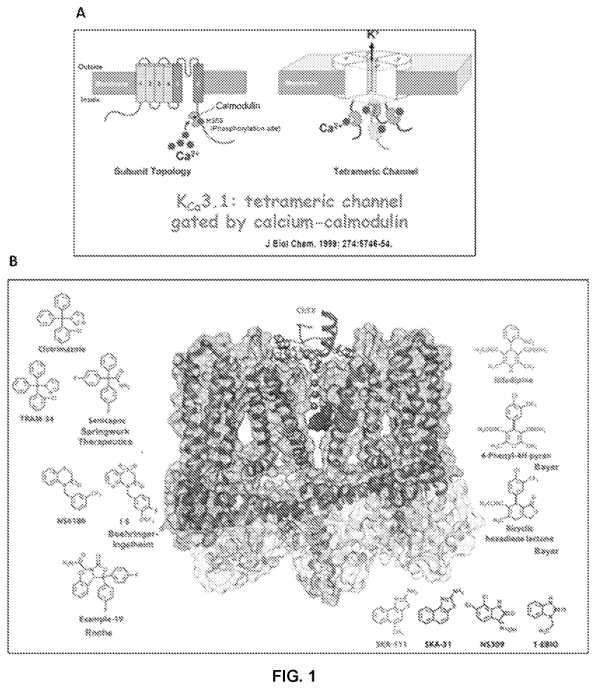

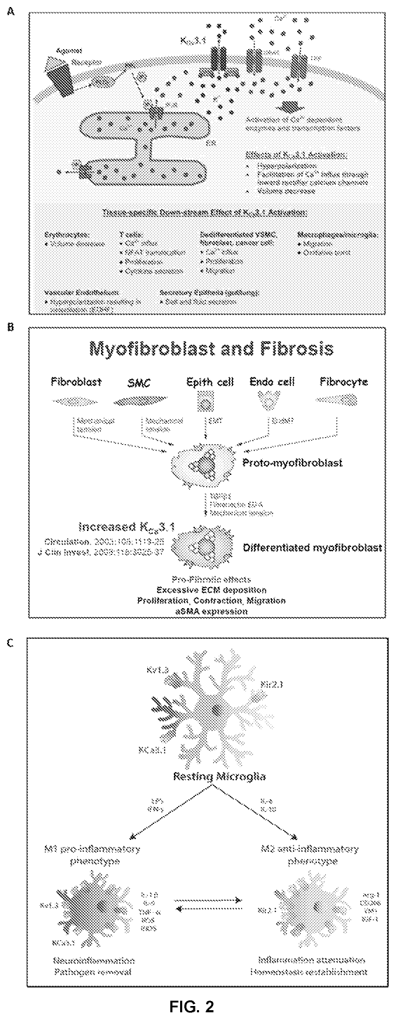

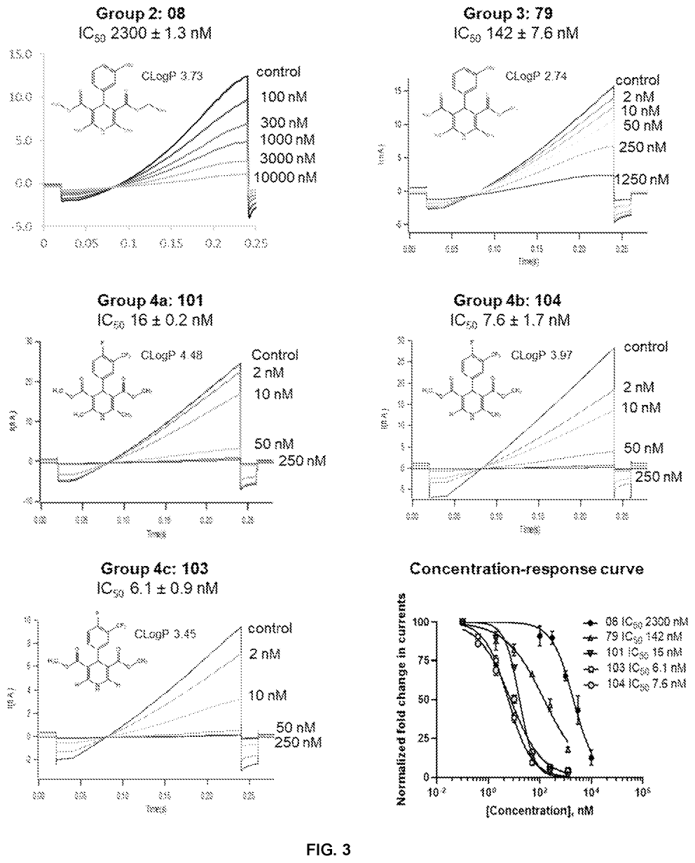

### DESCRIPTION

As noted above, it has been surprisingly found that a series of phenyl-dihydropyridines display unexpectedly good properties that overcome one or more of the problems identified hereinbefore. Thus, in a first aspect of the invention, there is provided a compound of formula I:

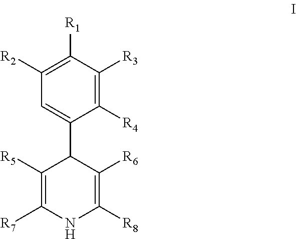

where:
R₁ is selected from H, halo, CF₃, CN or NO₂;
R₂ and R₃ are independently selected from H, halo, CH₃, CF₃, CN or NO₂;
R₄ is selected from H, halo, CN or CF₃;
R₅ and R₆ are independently selected from R₉ₐC(O)O—, R_{9b}OC(O)—, R_{9c}C(O)NR_{d}—,
R₉ₑR_{9f}NC(O)—, or an alkyl ketone having from 1 to 10 carbon atoms, which carbon atoms are branched or unbranched and are unsubstituted or substituted by one of more substituents selected from halo, and NO₂;
R₇ and R₈ are independently selected from H, NR₁₀ₐR_{10b}, OR_{10c} or C₁ to C₃ alkyl which is unsubstituted or substituted by one or more substituents selected from halo, or
   one of the pair of R₅ and R₇ or R₆ and R₈, together with the carbon atoms that they are attached to, form a 4- to 14-membered ring system that is carbocyclic or heterocyclic and which is unsubstituted or substituted by one or more substituents selected from halo, ═O, —OC(O)R_{10d}, —(O)COR₁₀ₑ, and C₁ to C₆ alkyl;
R₉ₐ to R_{9f} and R₁₀ₐ to R₁₀ₑ are independently selected from H and C₁ to C₃ alkyl which is unsubstituted or substituted by one or more substituents selected from halo, or pharmaceutically acceptable salts and/or solvates thereof.

The word “comprising” refers herein may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise.

References herein (in any aspect or embodiment of the invention) to compounds of formula I includes references to such compounds per se, to tautomers of such compounds, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. Lascorbic), L-aspartic, benzoic, 4-acetamidobenzoic, butanoic, (+) camphoric, camphorsulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula I are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

“Pharmaceutically functional derivatives” of compounds of formula I as defined herein includes ester derivatives and/or derivatives that have, or provide for, the same biological function and/or activity as any relevant compound of the invention. Thus, for the purposes of this invention, the term also includes prodrugs of compounds of formula I.

The term “prodrug” of a relevant compound of formula I includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

Prodrugs of compounds of formula I may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula I wherein a hydroxyl, amino, sulfhydryl, carboxyl or carbonyl group in a compound of formula I is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. I-92, Elsevier, New York-Oxford (1985).

Compounds of formula I, as well as pharmaceutically acceptable salts, solvates and pharmaceutically functional derivatives of such compounds are, for the sake of brevity, hereinafter referred to together as the “compounds of formula I”.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention.

Compounds of formula I may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

The term “halo”, when used herein, includes references to fluoro, chloro, bromo and iodo.

When recited herein, when a 4- to 14-membered ring system that is carbocyclic or heterocyclic is mentioned herein, it may contain from one to three rings. For the avoidance of doubt, the 4- to 14-membered ring system that is carbocyclic or heterocyclic mentioned herein may be formed in part using atoms that are already part of a ring, which should not be counted to the total number of rings in the 4- to 14-membered ring system. The 4- to 14-membered ring system may be a 6- to 10-membered ring system, such as a 6- to 8-membered ring system, which may be monocyclic or bicyclic.

Unless otherwise stated, the term “aryl” when used herein includes C₆₋₁₄ (such as C₆₋₁₀) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Embodiments of the invention that may be mentioned include those in which aryl is phenyl.

Unless otherwise stated, the term “alkyl” refers to an unbranched or branched, acyclic or cyclic, saturated or unsaturated (so forming, for example, an alkenyl or alkynyl)hydrocarbyl radical, which may be substituted or unsubstituted (with, for example, one or more halo atoms). Where the term “alkyl” refers to an acyclic group, it is preferably C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₅₋₁₀ (e.g. C₅₋₇) cycloalkyl.

Unless otherwise specified herein, a “heterocyclic ring system” may be 4- to 14-membered, such as a 5- to 10-membered (e.g. 6- to 10-membered), heterocyclic group that may be aromatic (i.e. a heteroaryl group), fully saturated or partially unsaturated, and which contains one or more heteroatoms selected from O, S and N, which heterocyclic group may comprise one or two rings. Examples of hetereocyclic ring systems that may be mentioned herein include, but are not limited to azetidinyl, dihydrofuranyl (e.g. 2,3-dihydrofuranyl, 2,5-dihydrofuranyl), dihydropyranyl (e.g. 3,4-dihydropyranyl, 3,6-dihydropyranyl), 4,5-dihydro-1H-maleimido, dioxanyl, dioxolanyl, furanyl, furazanyl, hexahydropyrimidinyl, hydantoinyl, imidazolyl, isothiaziolyl, isoxazolidinyl, isoxazolyl, morpholinyl, 1,2- or 1,3-oxazinanyl, oxazolidinyl, oxazolyl, piperidinyl, piperazinyl, pyranyl, pyrazinyl, pyridazinyl, pyrazolyl, pyridinyl, pyrimidinyl, pyrrolinyl (e.g. 3-pyrrolinyl), pyrrolyl, pyrrolidinyl, pyrrolidinonyl, 3-sulfolenyl, sulfolanyl, tetrahydrofuranyl, tetrahydropyranyl, tetrahydropyridinyl (e.g. 3,4,5,6-tetrahydropyridinyl), 1,2,3,4-tetrahydropyrimidinyl, 3,4,5,6-tetrahydropyrimidinyl, tetrahydrothiophenyl, tetramethylenesulfoxide, tetrazolyl, thiadiazolyl, thiazolyl, thiazolidinyl, thienyl, thiophenethyl, triazolyl and triazinanyl.

Unless otherwise specified herein, a “carbocyclic ring system” may be 4- to 14-membered, such as a 5- to 10-membered (e.g. 6- to 10-membered, such as a 6-membered or 10-membered), carbocyclic group that may be aromatic, fully saturated or partially unsaturated, which carbocyclic group may comprise one or two rings. Examples of carbocyclic ring systems that may be mentioned herein include, but are not limited to cyclobutyl, cyclopentyl, cyclohexyl, cyclooctyl, phenyl, naphthyl, decalinyl, tetralinyl, bicyclo[4.2.0]octanyl, and 2,3,3a,4,5,6,7,7a-octahydro-1H-indanyl. Particularly preferred carbocyclic groups include phenyl, cyclohexyl and naphthyl.

The term “heteroaryl” when used herein refers to an aromatic group containing one or more heteroatom(s) (e.g. one to four heteroatoms) preferably selected from N, O and S (so forming, for example, a mono-, bi-, or tricyclic heteroaromatic group). Heteroaryl groups include those which have between 5 and 14 (e.g. 10) members and may be monocyclic, bicyclic or tricyclic, provided that at least one of the rings is aromatic. However, when heteroaryl groups are bicyclic or tricyclic, they are linked to the rest of the molecule via an aromatic ring. Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Particularly preferred heteroaryl groups include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, thiophenyl, pyranyl, carbazolyl, acridinyl, quinolinyl, benzoimidazolyl, benzthiazolyl, purinyl, cinnolinyl and pterdinyl. Particularly preferred heteroaryl groups include monocylic heteroaryl groups.

Further embodiments of the invention that may be mentioned include those in which the compound of formula I is isotopically labelled. However, other, particular embodiments of the invention that may be mentioned include those in which the compound of formula I is not isotopically labelled.

The term “isotopically labelled”, when used herein includes references to compounds of formula I in which there is a non-natural isotope (or a non-natural distribution of isotopes) at one or more positions in the compound. References herein to “one or more positions in the compound” will be understood by those skilled in the art to refer to one or more of the atoms of the compound of formula I. Thus, the term “isotopically labelled” includes references to compounds of formula I that are isotopically enriched at one or more positions in the compound.

The isotopic labelling or enrichment of the compound of formula I may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷Cl, ⁷⁷Br, ⁸²Br and ¹²⁵I.

When the compound of formula I is labelled or enriched with a radioactive or nonradioactive isotope, compounds of formula I that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

Embodiments of the invention that may be mentioned include those in which the compounds of formula I selectively inhibit the K_{Ca}3.1 calcium-activated potassium channel subtype.

When used herein in relation to inhibition of the K_{Ca}3.1 calcium-activated potassium channel, the terms “selective” and “selectivity” includes references to the binding of a compound to the K_{Ca}3.1 with an IC₅₀ value that is at least 10-fold lower (e.g. at least 20-, 50-, 100-, 500- or 1000-fold lower) than the IC₅₀ value determined for the binding of the same compound to the Caᵥ1.2 calcium-activated potassium channel sub-type at the same temperature (e.g. room temperature, such as 298 K).

Embodiments of the invention that may also be mentioned include those in which the compounds of formula I are selective inhibitors of the K_{Ca}3.1 calcium-activated potassium channel.

When used herein in relation to inhibition of the K_{Ca}3.1 calcium-activated potassium channel, the terms “selective” and “selectivity” includes references to the binding of a compound to the K_{Ca}3.1 calcium-activated potassium channel with an IC₅₀ value that is at least 10-fold lower (e.g. at least 20-, 50-, 100-, 500- or 1000-fold lower) than the IC₅₀ value determined for the binding of the same compound to another calcium-activated potassium channel subtype (e.g. the Caᵥ1.2 calcium-activated potassium channel sub-type) at the same temperature (e.g. room temperature, such as 298 K). Selectivity for the K_{Ca}3.1 calcium-activated potassium channel can be over one other calcium-activated potassium channel subtypes but, in certain embodiments of the invention, is over two or more (e.g. all other) calcium-activated potassium channel subtypes.

In the second aspect of the invention, there is provided compound or derivatives thereof as described in the first aspect for use as a K_{Ca}3.1 channel inhibitor. Embodiments of the invention that may be mentioned include those in which the compounds of formula I selectively inhibit the K_{Ca}3.1 channel.

When used herein in relation to inhibition of the K_{Ca}3.1 channel, the terms “selective” and “selectivity” includes references to the binding of a compound to the K_{Ca}3.1 channel with an IC₅₀ value that is at least 10-fold lower (e.g. at least 20-, 50-, 100-, 500- or 1000-fold lower) than the IC₅₀ value determined for the binding of the same compound to one or more of the following channels: K_{Ca}1.1 channel, K_{Ca}2.2 channel, K_{Ca}2.3 channel, K_{V}1.1 channel, K_{V}1.2 channel, K_{V}1.3 channel, K_{V}1.4 channel, K_{V}1.5 channel, K_{V}1.7 channel, K_{V}3.1 channel, K_{V}4.2 channel, K_{V}11.1 channel at the same temperature (e.g. room temperature, such as 298 K). Selectivity for the K_{Ca}3.1 channel can be over one other calcium channel subtype and/or voltage gated channel subtype but, in certain embodiments of the invention, is over two or more (e.g. all other) calcium channel subtypes and voltage gated channel subtypes.

In embodiments of the invention, one or more of the following may apply:
(Ai) R₁ may be selected from H, F, Cl, Br, CF₃ or NO₂;
(Aii) R₂ and R₃ may independently be selected from H, F, Cl, Br, CH₃, CF₃ or NO₂;
(Aiii) R₄ may be selected from H, F, Cl, Br, or CF₃;
(Aiv) R₅ and R₆ may independently be selected from R₉ₐC(O)O—, R_{9b}OC(O)—, or an alkyl ketone having from 1 to 10 carbon atoms, which carbon atoms are branched or unbranched and are unsubstituted or substituted by one of more substituents selected from Cl, F, and NO₂;
(Av) R₇ and R₈ may independently be selected from H or C₁ to C₃ alkyl which is unsubstituted or substituted by one or more substituents selected from F and Cl, or
   one of the pair of R₅ and R₇ or R₆ and R₈, together with the carbon atoms that they are attached to, may form a 4- to 10-membered ring system that is carbocyclic or heterocyclic and which is unsubstituted or substituted by one or more substituents selected from F, Cl, ═O, and C₁ to C₆ alkyl;
(Avi) R₉ₐ to R_{9f} and R₁₀ₐ to R₁₀ₑ are independently selected from H and C₁ to C₃ alkyl which is unsubstituted or substituted by one or more substituents selected from F and Cl.

In particular embodiments that may be mentioned herein, one or more of the following may apply:
R₁ is selected from H, F, Cl, or CF₃;
R₂ and R₃ are independently selected from H, F, Cl, or CF₃;
R₄ is selected from H, F, Cl, or CF₃;
R₅ and R₆ are independently selected from R_{9b}OC(O)—, an alkyl ketone having from 1 to 3 carbon atoms, which carbon atoms are unsubstituted or substituted by one of more substituents selected from Cl and F;
R₇ and R₈ are independently selected from H, or methyl which is unsubstituted or substituted by one or more substituents selected from F and Cl.

In more particular embodiments of the invention that may be mentioned herein one or more of the following may apply
R₁ is selected from H, F, or Cl;
R₂ is selected from CF₃ or, more particularly, H or F;
R₃ is selected from H or CF₃;
R₄ is H;
R₅ and R₆ are independently selected from CH₃OC(O)— or propan-2-onyl (e.g. R₅ and R₆ are both CH₃OC(O)—);
R₇ and R₈ are independently selected from H and CH₃.

In particular embodiments of the invention that may be mentioned herein at least one of R₇ and R₈ is H. For example, both of R₇ and R₈ may be H. In alternative embodiments that may be mentioned herein:
R₇ may be H and R₈ may be CH₃;
R₇ and R₈ may both be CH₃; or
R₇ may be CH₃ and R₈ may be H.

The compound of formula I, or a salt and/or solvate thereof, may be selected from one or more of the compounds from the list:

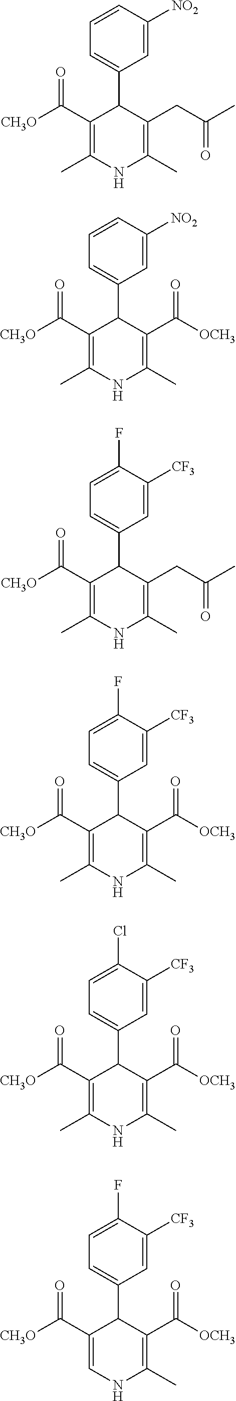

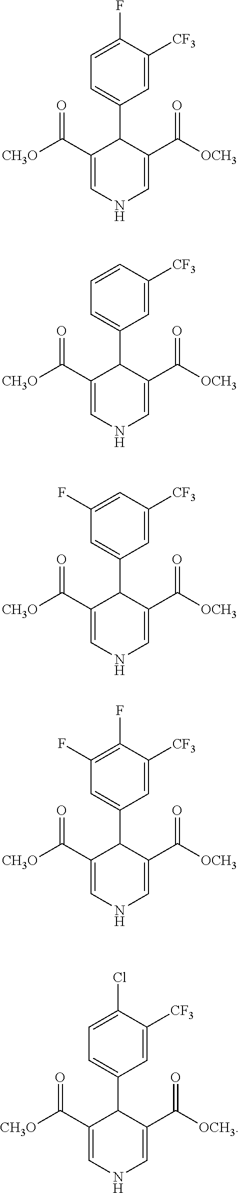

More particularly, the compound of formula I, or a salt and/or solvate thereof, may be selected from one or more of the compounds from the list:

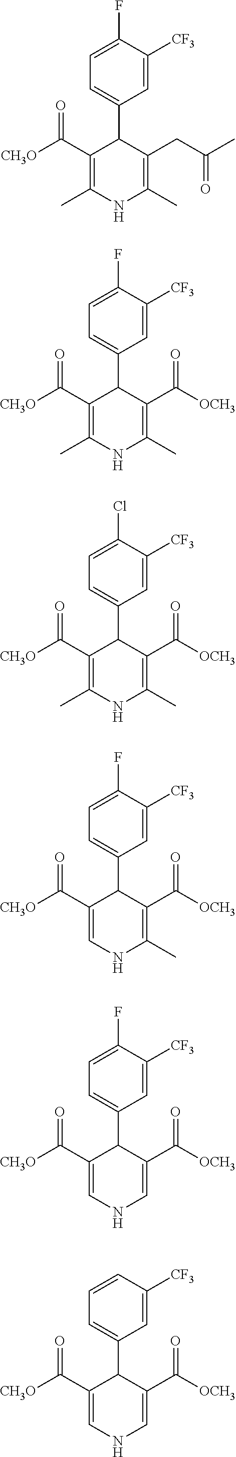

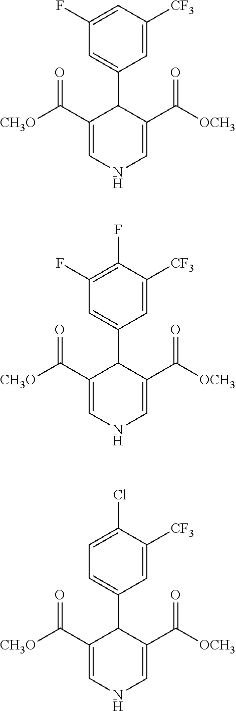

Yet more particularly, the compound of formula I, or a salt and/or solvate thereof, may be selected from one or more of the compounds from the list:

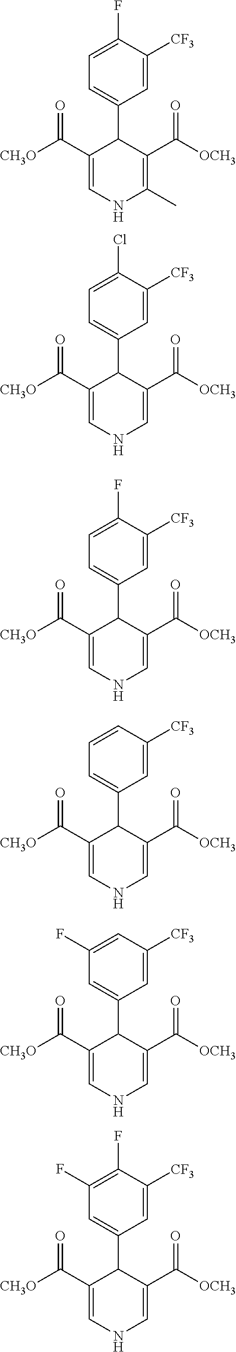

Still more particularly, the compound of formula I, or a salt and/or solvate thereof, may be selected from one or more of the compounds from the list:

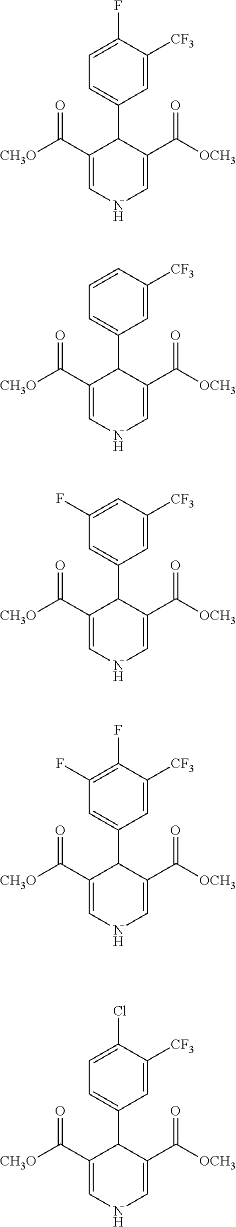

For example the compound of formula I, or a salt and/or solvate thereof, may be

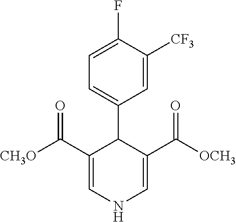

As will be appreciated, the compounds of the current invention may be suitable for treating a subject. As such, in a further aspect of the invention, there is provided a pharmaceutical composition comprising a compound of formula I, or salts and solvates thereof, as described hereinbefore in combination with one or more of a pharmaceutically acceptable carrier, adjuvant, or vehicle.

Compounds of formula I may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

Compounds of formula I will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995).

For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of compound of formula I in any pharmaceutical formulation used in accordance with the present invention will depend on various factors, such as the severity of the condition to be treated, the particular patient to be treated, as well as the compound(s) which is/are employed. In any event, the amount of compound of formula I in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, compounds of formula I may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

As will be appreciated, a further aspect of the invention relates to a compound of formula I, or a salt and/or solvate thereof, as described hereinbefore, for use in medicine.

Thus, further aspects of the invention relate to the following.
(a) A compound of formula I, or salts and solvates thereof, as hereinbefore defined, for use in the treatment or prevention of a disease condition that is associated with increased KCa3.1 or altered activity.
(b) Use of a compound of formula I, or salts and solvates thereof, as hereinbefore defined, for the preparation of a medicament for the treatment or prevention of a disease condition that is associated with increased KCa3.1 or altered activity.
(c) A method of treatment of a disorder or condition that is associated with increased K_{Ca}3.1 or altered activity, which method comprises the administration of an effective amount of a compound of formula I, or salts and solvates thereof, as hereinbefore defined to a subject in need thereof.

In various embodiments of the invention, the disease condition associated with increased or altered K_{Ca}3.1 activity may be selected from one or more of leukaemia or, more particularly, inflammatory bowel diseases (IBD), fibrotic diseases (lung, liver, renal, cardiac, conjunctival, corneal), non-alcoholic fatty liver disease (NAFLD), nonalcoholic steatohepatitis (NASH), gliomas (glioblastoma), lung cancer, pancreatic cancer, hepatocellular carcinoma, ovarian cancer, colorectal cancer, cystic fibrosis, diabetic renal disease, glomerulonephritis, bone resorption, inflammatory arthritis, multiple sclerosis, atherosclerosis, restenosis following angioplasty, in-stent neo-atherosclerosis, stroke, traumatic brain injury, Alzheimer's disease, hereditary xerocytosis, sickle cell anemia, asthma, allergic rhinitis, microglial activation, nitric oxide-dependent neurodegeneration, neuro-oncological diseases and orphan red blood cell disorders. For example, the disease condition associated with increased or altered K_{Ca}3.1 activity may be selected from one or more of leukaemia, or more particularly inflammatory bowel diseases (IBD), fibrotic diseases (lung, liver, renal, cardiac, conjunctival, corneal), non-alcoholic fatty liver disease (NAFLD), nonalcoholic steatohepatitis (NASH), gliomas (glioblastoma), lung cancer, pancreatic cancer, hepatocellular carcinoma, ovarian cancer, colorectal cancer, cystic fibrosis, diabetic renal disease, glomerulonephritis, bone resorption, inflammatory arthritis, multiple sclerosis, atherosclerosis, restenosis following angioplasty, in-stent neo-atherosclerosis, stroke, traumatic brain injury, Alzheimer's disease, hereditary xerocytosis, sickle cell anemia, asthma, allergic rhinitis, microglial activation and nitric oxide-dependent neurodegeneration.

In particular embodiments that may be mentioned herein, the disease condition associated with increased or altered K_{Ca}3.1 activity may be stroke.

As will be appreciated, the compound or derivatives of formula I disclosed herein has advantages of potency, selectivity for K_{Ca}3.1 channel, oral bioavailability, excellent brain penetration, and well-tolerability in repeat-dose toxicological studies in rodents. Further, the compound or derivatives of formula I disclosed herein has been found to be effective in treating stroke which is a disease condition associated with increased or altered K_{Ca}3.1 activity.

The aspects of the invention described herein (e.g. the above-mentioned compounds, combinations, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, or may have other useful pharmacological properties over, similar compounds, combinations, methods (treatments) or uses known in the prior art for use in the treatment of those conditions or otherwise.

Compounds of formula I may be synthesised using known techniques as demonstrated in the below Examples. Other compounds may be prepared by analogous methods. Compounds of the invention may be isolated from their reaction mixtures using conventional techniques (e.g. recrystallisation, column chromatography, preparative HPLC, etc.).

### EXAMPLES

Materials

Ammonium carbonate ((NH₄)₂CO₃), acetic acid, ammonium acetate (NH₄OAc), dicyclohexyl carbamide, 4-dimethyl aminopyridine, n,o-dimethyl hydroxyl amine HCl, 2-(1H-benzotriazole-1-YL)-1,1,3,3-tetramethyluronium hexafluorophosphate (HBTU), ethyl chloroformate, methyl acetoacetate, piperidine and sodium meta per iodate were purchased from Avra Synthesis Pvt Ltd. Sodium chloride, potassium chloride, magnesium chloride, calcium chloride, ethylene glycol-bis(β-aminoethyl ether)-N,N,N′,N′-tetraacetic acid (EGTA), D-glucose, guanosine 5′-triphosphate sodium salt hydrate(Na₂-GTP), Adenosine 5′-triphosphate magnesium salt (Mg-ATP), Ketoconazole, Quinidine, Sulphaphenazolefor, Nootkatone, Verapamil, Warfarin, Naltrexone, Loperamide hydrochloride, Phenacetin, Reserpine, R-Nicotinamide adenine dinucleotide 2′-phosphate reduced tetrasodium salt hydrate (NADPH), N-Methyl-2-pyrrolidone (NMP), Solutol HS-15, polyethylene glycol 400 (PEG-400), Tocophersolan (TPGS), manganese oxide and dichloromethane were purchased from Sigma-Aldrich. Osmium (VIII) oxide was purchased from Chempure Pte Ltd. Methyl propiolate, 4-chloro-3-(trifluoromethyl) benzaldehyde, 3,4-difluoro-5-(trifluoromethyl) benzoic acid, 3-fluoro-5-(trifluoromethyl) benzaldehyde, potassium vinyltrifluoroborate (95%), 5-bromo-2-fluoro-3-(trifluoromethyl) benzoic acid and 4-fluoro-3-(trifluoromethyl) benzaldehyde were purchased from Combi-Blocks Inc. 1,1′-bis-(diphenylphosphino)-ferrocene]-palladous dichloride was purchased from Hindustan Platinum Pte Ltd. Chloroform were purchased from SAI Enterprises. Isopropylmagnesium chloride-lithium chloride complex, 1.3 M solution in THF, hydrogen chloride 4 mol in 1,4-dioxane, and methylmagnesiumbromide 3 M solution in diethyl ether were purchased from Sainor Laboratories Pte. Ltd. N,N,N,N-tetramethylguanidinium azide was purchased from SelectLab Chemicals GmbH. Methyl propiolate, tetraethylammonium chloride (TEA-Cl), and 3-(trifluoromethyl) benzaldehyde (97%) were purchased from TCI Chemicals (India) Pte. Ltd. N-2-hydroxyethylpiperazine-N-2-ethane sulfonic acid (HEPES) was purchased from Thermo Fisher Scientific. Furafylline was purchased from BD Gentest. Formic acid was purchased from Honeywell Research Chemicals. Acetonitrile (ACN) was purchased from Avantor. Tolbutamide was purchased from Supelco. 2,3,5-triphenyltetrazolium chloride (TCC) was purchased from Vicmed Biotechnology. Edaravone was purchased from TargetMol. All other chemicals and solvents were purchased from industrial chemical suppliers and they were directly used without further purification.

Analytical Techniques

¹H NMR Spectroscopy

All ¹H NMR spectra were recorded on 400 MHz (Bruker) and 500 MHz (Agilent) NMR spectrometers. All chemical shifts are given as δ value with reference to Tetra methyl silane (TMS) as an internal standard.

Liquid Chromatography-Mass Spectrometry (LC-MS)

For toxicology studies, LC-MS/MS AB SCIEX API-4000 Triple Quadrapole instrument coupled with Waters UPLC system was used. For cytochrome P450 enzymes inhibition assay and plasma protein binding assay, mass spectrometer API-4000 (Applied Biosystems) coupled with LC SIL-HTc (Shimadzu) were used. For microsomal stability study, mass spectrometer TSQ Quantum Ultra (Thermo Scientific) coupled with LC SIL-HTc (Shimadzu) was used. For compound synthesis analysis, LCMS (SQD)-2010EV (Shimadzu), LCMS (SQD)-1200 series LC/G6125B-MS (Agilent) and UPLC/MS (SQD) (Waters) were used.

High-Performance Liquid Chromatography (HPLC)

For aqueous solubility assay, Waters Alliance 2690 HPLC was used. For compound synthesis analysis, HPLC 2010CHT (Shimadzu) was used.

### Example 1

The dihydropyridines studied in this work are classified into four groups and are presented in Table 1. Group 4 is further divided into three sub-groups (4a, 4b, 4c) based on the moieties at R7 and R8.

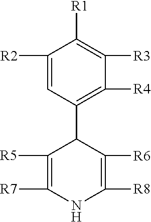

TABLE 1
Four groups of inhibitors and their chemical substitutions are shown.

| | | | | | | | | |
|---|---|---|---|---|---|---|---|---|
| Name | R1 | R2 | R3 | R4 | R5 | R6 | R7 | R8 |

Group 1

| | | | | | | | | |
|---|---|---|---|---|---|---|---|---|
| 1 | — | — | — | Cl | methyl acetate | methyl acetate | CH₃ | 2-ethoxyethan- |
| | | | | | | | | 1-amine |
| 2 | — | — | — | Cl | methyl acetate | ethyl acetate | CH₃ | 2-ethoxyethan- |
| | | | | | | | | 1-amine |
| 3 | — | — | NO₂ | — | methyl acetate | 2-(4- | CH₃ | CH₃ |
| | | | | | | benzhydrylpiperazin-1- | | |
| | | | | | | yl)ethyl acetate | | |
| 4 | — | — | NO₂ | — | isopropyl acetate | 2-methoxyethyl acetate | CH₃ | CH₃ |
| 5 | — | — | NO₂ | — | isopropyl acetate | methyl acetate | CH₃ | N |
| 6 | — | — | NO₂ | — | 2-methoxyethyl | 2-methoxyethyl acetate | CH₃ | CH₃ |
| | | | | | acetate | | | |
| 72 | — | — | NO₂ | — | methyl acetate | cyclopent-2-en-1-one | CH₃ | CH₃ |
| 77 | — | — | NO₂ | — | methyl acetate | 3,4-dihydro-2H- | CH₃ | H |
| | | | | | | thiopyran 1,1-dioxide | | |
| 76 | — | — | NO₂ | — | methyl acetate | 2,3-dihydrothiophene | CH₃ | CH₃ |
| | | | | | | 1,1-dioxide | | |
| 87 | — | — | NO₂ | — | cyclohex-2-en-1-one | cyclohex-2-en-1-one | CH₃ | H |

Group 2

| | | | | | | | | |
|---|---|---|---|---|---|---|---|---|
| 7 | — | — | — | NO₂ | methyl acetate | methyl acetate | CH₃ | CH₃ |
| 73 | — | — | NO₂ | — | methyl acetate | cyclohex-2-en-1-one | CH₃ | H |
| 75 | — | — | NO₂ | — | methyl acetate | (methylsulfonyl)methane | CH₃ | CH₃ |
| 8 | — | — | NO₂ | — | methyl acetate | ethyl acetate | CH₃ | CH₃ |
| 56 | — | — | — | — | methyl acetate | propan-2-one | CH₃ | CH₃ |
| 85 | — | — | NO₂ | — | propan-2-one | propan-2-one | CH₃ | CH₃ |
| 105 | F | methyl | CF₃ | — | methyl acetate | methyl acetate | CH₃ | CH₃ |
| | | acetate | | | | | | |
| 106 | F | Acetamide | CF₃ | — | methyl acetate | methyl acetate | CH₃ | CH₃ |

Group 3

| | | | | | | | | |
|---|---|---|---|---|---|---|---|---|
| 79 | — | — | NO₂ | — | methyl acetate | propan-2-one | CH₃ | CH₃ |
| 84 | — | — | NO₂ | — | methyl acetate | methyl acetate | CH₃ | CH₃ |

Group 4a*

| | | | | | | | | |
|---|---|---|---|---|---|---|---|---|
| 57 | F | — | CF₃ | — | methyl acetate | propan-2-one | CH₃ | CH₃ |
| 101 | F | — | CF₃ | — | methyl acetate | methyl acetate | CH₃ | CH₃ |
| 102 | Cl | — | CF₃ | — | methyl acetate | methyl acetate | CH₃ | CH₃ |

Group 4b*

| | | | | | | | | |
|---|---|---|---|---|---|---|---|---|
| 104 | F | — | CF₃ | — | methyl acetate | methyl acetate | H | CH₃ |

Group 4c*

| | | | | | | | | |
|---|---|---|---|---|---|---|---|---|
| 103 | F | — | CF₃ | — | methyl acetate | methyl acetate | H | H |
| 108 | H | — | CF₃ | — | methyl acetate | methyl acetate | H | H |
| 109 | H | F | CF₃ | | methyl acetate | methyl acetate | H | H |
| 110 | F | F | CF₃ | | methyl acetate | methyl acetate | H | H |
| 113 | Cl | — | CF₃ | | methyl acetate | methyl acetate | H | H |

The synthesis of Group 4b and 4c analogues are shown below. All these compounds are new chemical entities based on a search of the SciFinder database.

### Dimethyl 4-(4-fluoro-3-(trifluoromethyl) phenyl)-1, 4-dihydropyridine-3, 5-dicarboxylate (103)

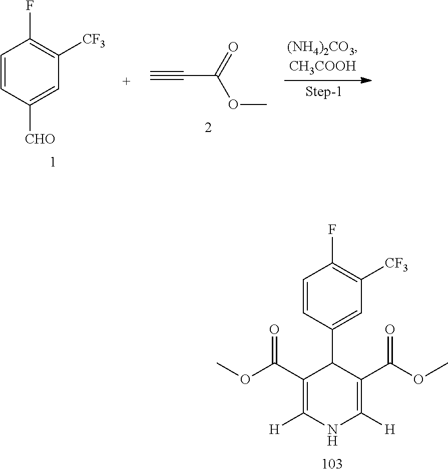

To a stirred solution of 4-fluoro-3-(trifluoromethyl)benzaldehyde (1, 80 g, 416.42 mmol, 1.0 eq) in acetic acid (800 mL) cooled to 0° C., methyl propiolate (2, 70 g, 832.84 mmol, 2.0 eq) and (NH₄)₂CO₃ (40 g, 416.42 mmol, 1.0 eq) were added and the reaction mixture was stirred at 70° C. for 16 h. After complete reaction (monitored by thin layer chromatograph (TLC)), the reaction mixture was diluted with cold water (1 L), then extracted with ethyl acetate (EtOAc, 2×1 L). The organic phases were washed with saturated NaHCO₃ solution (2 L) and brine solution (2 L), dried over anhydrous Na₂SO₄, filtered and concentrated to obtain the crude product. The crude product was triturated using hexane (800 mL), decant then followed by crystallization with MeOH (50 mL) and filtration to obtain 103 (44 g, 29.4%) as a pale yellow solid. TLC: 50% EtOAc/Heptane (R_{f}: 0.3).

¹H NMR (DMSO-d₆, 400 MHz): δ 9.32 (br s, 1H), 7.55-7.50 (m, 1H), 7.48-7.44 (m, 1H), 7.43-7.37 (m, 3H), 4.80 (s, 1H), 3.55 (s, 6H).

LC-MS: 99.59%, m/z=358.0 [M−H]⁻ (Column: EVO-C18 (3.0×50 mm, 2.6 μm); Rₜ: 2.83 min; A: 2.5 mM NH₄OAc in water, B: acetonitrile (CAN) T/B %: 0.01/5, 3/90, 5/90, 5.5/5, 6/5; Temperature: 50° C.; and Flow Rate: 0.8 mL/min).

HPLC: 99.94% (Column: X-SELECT CSH C-18 (4.6×150 mm, 3.5 μm); Rₜ: 10.20 min; A: 5.0 mM NH₄OAc in water, B: ACN T/B %: 0.01/20, 12/90, 16/90; and Flow Rate: 1.0 mL/min).

### Dimethyl 4-(4-fluoro-3-(trifluoromethyl)phenyl)-2-methyl-1,4-dihydropyridine-3,5-dicarboxylate (104)

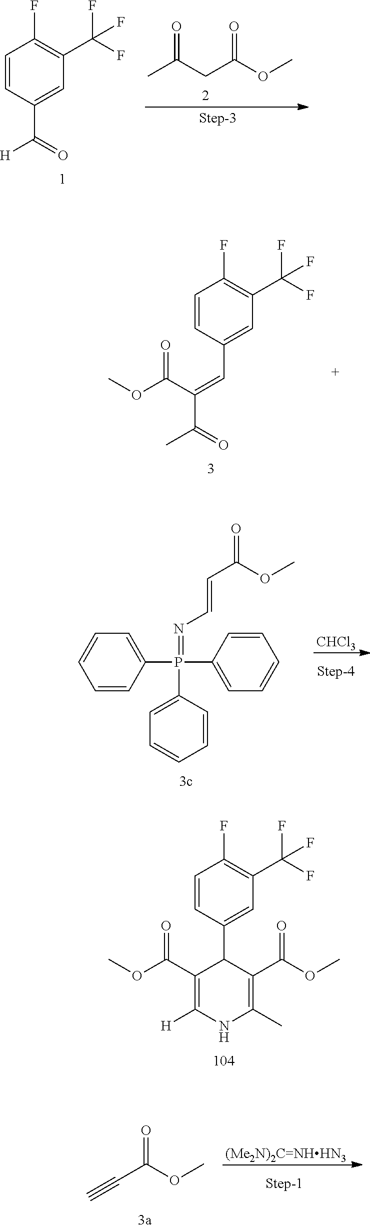

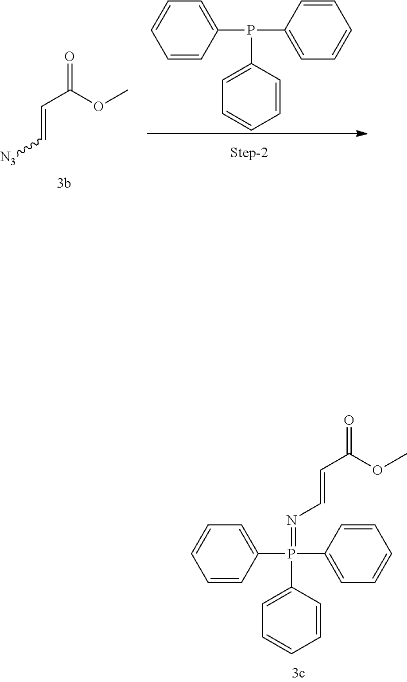

### Methyl 3-azidoacrylate (3b)

To a stirred solution of methyl propiolate (3a, 2.0 g, 23.80 mmol, 1.0 eq) in chloroform (40 mL), N,N,N,N-Tetramethyl guanidinium azide (4.0 g, 26.19 mmol, 1.1 eq) was added, and the reaction was stirred at room temperature (RT) for 16 h. After complete reaction (monitored by TLC), the reaction mixture was concentrated at 300 mbar in a 35° C. water bath. The crude product was distilled first with a house vacuum in a 35° C. water bath, which removed most of the remaining chloroform. The vacuum pump was connected to give a vacuum, and the crude product was distilled to obtain 3b as an off white solid (1.5 g, crude). The crude product was directly used in the next reaction without further purification. TLC: 20% EtOAc/heptane (R_{f}: 0.5).

### Methyl (E)-3-((triphenyl-15-phosphaneylidene)amino)acrylate (3c)

To a stirred solution of methyl 3-azidoacrylate (1.5 g, 11.81 mmol, 1.0 eq) in DCM (50 mL), triphenylphosphine (3.0 g, 11.80 mmol, 1.0 eq) was added and the reaction mixture was stirred at RT for 16 h. After complete reaction (monitored by TLC), the reaction mixture was directly evaporated under vacuum to obtain 3c as an off white solid (1.0 g, crude). TLC: 20% EtOAc/heptane (R_{f}: 0.5).

### Methyl (Z)-2-(4-fluoro-3-(trifluoromethyl)benzylidene)-3-oxobutanoate (2)

To a stirred solution of 4-fluoro-3-(trifluoromethyl)benzaldehyde (1, 0.5 g, 2.60 mmol, 1.0 eq) in toluene (10 mL), piperdine (22.1 mg, 0.26 mmol, 0.1 eq) and acetic acid (15.6 mg, 0.26 mmol, 0.1 eq) were added at 0° C. and the reaction mixture was stirred at RT for 16 h. After complete reaction (monitored by TLC), the reaction mixture was diluted with cold water (20 mL), then extracted with DCM (2×50 mL). The organic phases are washed with brine solution (20 mL), dried over anhydrous Na₂SO₄, filtered and concentrated to obtain the crude product which was purified by combi-flash chromatography using 20-30% EtOAc in hexane as an eluent to give 2 as a pale yellow solid (0.5 g, 66.2%). TLC: 30% EtOAc/heptane (R_{f}: 0.2).

104

To a stirred solution of 2 (0.5 g, 1.72 mmol, 1.0 eq) in chloroform (20 mL), 3c (0.930 g, 2.58 mmol, 1.5 eq) was added and the reaction mixture was stirred at 40° C. for 6 h. The reaction progress was monitored by TLC and the product was worked up and purified by following the protocol for 2 above to give 104 as a pale yellow solid (0.2 g, 31%). TLC: 40% EtOAc/Heptane (R_{f}: 0.2).

¹H NMR (DMSO-d₆ 400 MHz): δ 9.36 (br s, 1H), 7.55-7.50 (m, 1H), 7.48-7.44 (m, 2H), 7.43-7.37 (m, 1H), 4.82 (s, 1H), 3.55 (s, 6H) 2.25 (s, 3H).

LC-MS: 99.91%, m/z=374.1 [M+H]⁺ (Column: EVO-C18 (3.0×50 mm, 2.6 μm); Rₜ: 2.8\8 min; A: 2.5 mM ammonium formate in water, B: ACN T/B %: 0.01/5, 3/90, 5/90, 5.5/5, 6/5; Temperature: 50° C.; and Flow Rate: 0.8 mL/min).

HPLC: 99.94% (Column: X-SELECT CSH C-18 (4.6×150 mm, 3.5 μm); Rₜ: 10.95 min; A: 5.0 mM ammonium acetate (NH₄OAc) in water, B: ACN T/B %: 0.01/20, 12/90, 16/90; and Flow Rate: 1.0 mL/min).

### Dimethyl 4-(3-(trifluoromethyl)phenyl)-1,4-dihydropyridine-3,5-dicarboxylate (108)

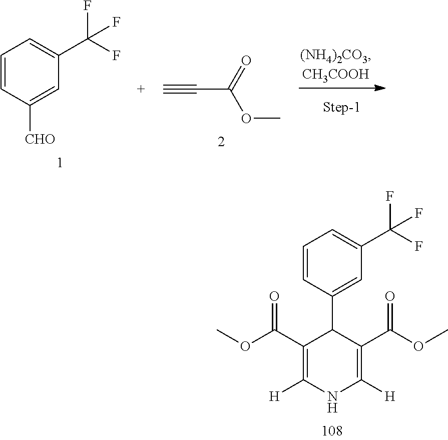

To a stirred solution of 3-(trifluoromethyl)benzaldehyde (1, 20 g, 114.92 mmol, 1.0 eq) in acetic acid (200 mL) cooled to 0° C., methyl propiolate (2, 20.45 mL, 229.85 mmol, 2.0 eq) and (NH₄)₂CO₃ (11 g, 114.92 mmol, 1.0 eq) were added and stirred at 80° C. for 16 h. After complete reaction (monitored by TLC), the reaction mixture was diluted with cold water (400 mL), then extracted with EtOAc (2×500 mL). The organic layers were washed with saturated NaHCO₃ solution (500 mL) and brine solution (500 mL), dried over anhydrous Na₂SO₄, filtered and concentrated to obtain the crude product which was purified by combi-flash chromatography using 30-40% EtOAc in hexane as an eluent to obtain 108 as a pale yellow solid (11.0 g, 28%). TLC: 40% EtOAc/Heptane (R_{f}: 0.2).

¹H NMR (DMSO-d₆, 400 MHz): δ 9.32 (br s, 1H), 7.55-7.50 (m, 3H), 7.48-7.44 (m, 1H), 7.43-7.37 (m, 2H), 4.80 (s, 1H), 3.55 (s, 6H).

LC-MS: 99.54%, m/z=340.0 [M−H]⁻ (Column: EVO-C18 (3.0×50 mm, 2.6 μm); Rₜ: 2.65 min; A: 2.5 mM NH₄OAc in water, B: ACN T/B %: 0.01/5, 3/90, 5/90, 5.5/5, 6/5; Temperature: 50° C.; and Flow Rate: 0.8 mL/min).

HPLC: 99.91% (Column: X-SELECT CSH C-18 (4.6×150 mm, 3.5 μm); Rₜ: 9.43 min; A: 5.0 mM NH₄OAc in water, B: ACN T/B %: 0.01/20, 12/90, 16/90; and Flow Rate: 1.0 mL/min).

### 4-(3-fluoro-5-(trifluoromethyl)phenyl)-1,4-dihydropyridine-3,5-dicarboxylate (109)

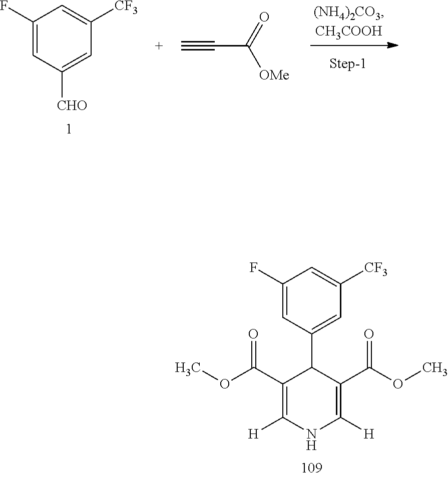

To a stirred solution of 3-fluoro-5-(trifluoromethyl)benzaldehyde (1, 0.100 g, 0.5208 mmol, 1.0 eq) and methyl propiolate (2, 0.085 g, 1.041 mmol, 2.0 eq) in acetic acid (10 mL), (NH₄)₂CO₃ (0.049 g, 0.5208, 1.0 eq) was added slowly at RT, and the reaction mixture was heated at 70° C. for 14 h. After complete consumption of the starting material (monitored by TLC), the reaction mixture was cooled to RT and slowly quenched with ice cold water (10 mL). The aqueous layer was extracted with DCM (15 mL×2). The combined organic layers were washed with brine (10 mL), dried over Na₂SO₄ and concentrated under reduced pressure to get the crude product which was purified by combi-flash column chromatography using 10-40% EtOAc in n hexane to afford 109 as a pale yellow solid (0.040 g, 22%). TLC: 40% EtOAc/hexane (R_{f}: 0.5).

¹H NMR (400 MHz, DMSO-d₆) δ 9.36 (brs, 1H), 7.40-7.50 (m, 3H), 7.32 (s, 1H), 7.26 (d, J=7.9 Hz, 1H), 4.81 (s, 1H), 3.53 (s, 6H).

LC-MS: 99.32%, m/z=360.0 [M+H]⁺ (Column: X-Select CSH C18 (3.0×50 mm, 2.5 μm); Rₜ: 1.941 min; A: 0.05% formic acid in water:ACN (95:05), B: 0.05% formic acid in ACN. Temperature: 50° C.; and Flow Rate: 1.2 mL/min).

HPLC: 99.45% (Column: X-SELECT CSH C-18 (4.6×150 mm, 3.5 μm); Rₜ: 8.48 min; A-0.1% formic acid in water, B: ACN T/B %: 0.01/5, 1.0/5, 8.0/100, 12.0/100, 14.0/5, 18.0/5 and Flow Rate: 1.0 mL/min).

### Dimethyl 4-(3,4-difluoro-5-(trifluoromethyl)phenyl)-1,4-dihydropyridine-3,5-dicarboxylate (110)

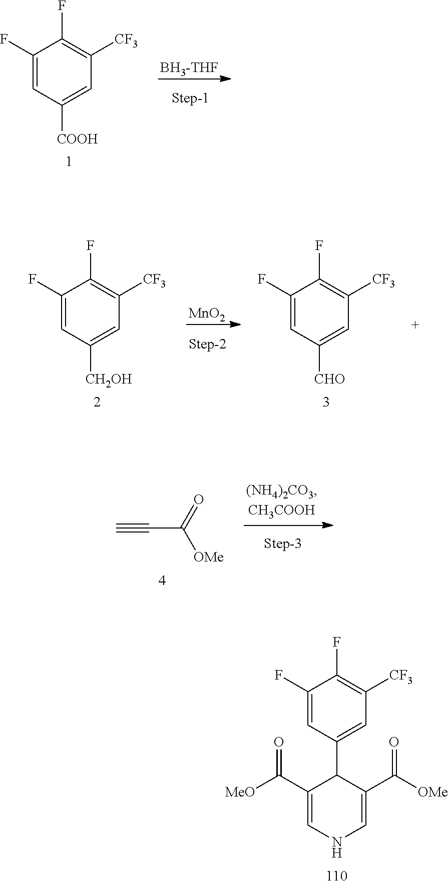

### (3,4-difluoro-5-(trifluoromethyl)phenyl)methanol (2)

To a stirred solution of 3,4-difluoro-5-(trifluoromethyl)benzoic acid (1, 0.500 g, 2.212 mmol) in THF (20 mL), BH₃:THF (6.6 mL, 6.637 mmol) was added slowly at 0° C. The reaction mixture was allowed to reach RT and was stirred for 12 h. After complete consumption of the starting material (monitored by TLC), the reaction mixture was quenched with 3 M HCl (30 mL) and the aqueous layer was extracted with EtOAc (20 mL). The organic layer was washed with brine (10 mL), dried over Na₂SO₄ and concentrated under reduced pressure to obtain 2 as a viscous colourless oil (0.300 g, 64%). The obtained crude product was directly used in the next reaction without further purification. TLC: 5% MeOH in DCM (R_{f}: 0.6).

¹H NMR (400 MHz, DMSO-d₆) δ 7.45-7.55 (m, 1H), 7.25 (s, 1H), 6.95 (s, 1H), 4.58-4.68 (s, 2H).

### 3,4-difluoro-5-(trifluoromethyl)benzaldehyde (3)

To a stirred solution of (3,4-difluoro-5-(trifluoromethyl)phenyl)methanol (2, 0.300 g, 1.415 mmol) in DCM (20 mL), MnO₂ (0.738 g, 8.490 mmol) was added slowly at RT and stirred for 12 h. After complete consumption of the starting material (monitored by TLC), the reaction mixture was filtered through a pad of Celite. The Celite pad was washed with DCM (5 mL) and the obtained filtrate was concentrated under reduced pressure to obtain 3 as a viscous colourless oil (0.200 g, 67%). The obtained crude product was directly used in the next reaction without further purification. TLC: 40% EtOAc/hexane (R_{f}: 0.8).

¹H NMR (400 MHz, DMSO-d₆) δ 9.88 (br. s, 1H), 7.73 (s, 1H), 7.45 (s, 1H).

110

To a stirred solution of 3,4-difluoro-5-(trifluoromethyl)benzaldehyde (3, 0.200 g, 0.9523 mmol) and methyl propiolate (2, 0.159 g, 1.904 mmol) in acetic acid (10 mL), (NH₄)₂CO₃ (0.091 g, 0.9523 mmol) was added slowly at RT and the reaction mixture was heated at 75° C. for 16 h. After complete consumption of starting material (monitored by TLC), the reaction mixture was cooled to RT and slowly quenched with ice cold water (10 mL). The aqueous layer was extracted with DCM (20 mL×2). The combined organic layers were washed with brine (10 mL), dried over Na₂SO₄ and concentrated under reduced pressure to get the crude product. The crude obtained was purified by combi-flash column chromatography using 0-40% EtOAc in n-hexane to afford 110 as a pale yellow solid (0.040 g, 11%). TLC: 40% EtOAc/hexane (R_{f}: 0.5).

¹H NMR (400 MHz, DMSO-d₆) δ 9.38 (br. s, 1H), 7.40-7.50 (m, 3H), 7.30 (s, 1H), 4.81 (s, 1H), 3.55 (s, 6H).

LC-MS: 99.64%, m/z=360.0 [M+H]⁺ (Column: X-Select CSH C18 (3.0×50 mm, 2.5 μm); Rₜ: 2.034 min; A: 0.05% formic acid in water:ACN (95:5), B: 0.05% formic acid in ACN; Temperature: 50° C.; and Flow Rate: 1.2 mL/min).

HPLC: 98.11% (Column; X-SELECT CSH C-18 (4.6×150 mm, 3.5 μm); Rₜ: 9.043 min; A-0.1% formic acid in water, B: ACN T/B %: 0.01/5, 1.0/5, 8.0/100, 12.0/100, 14.0/5, 18.0/5; and Flow Rate: 1.0 mL/min).

### Dimethyl 4-(4-chloro-3-(trifluoromethyl)phenyl)-1,4-dihydropyridine-3,5-dicarboxylate (113)

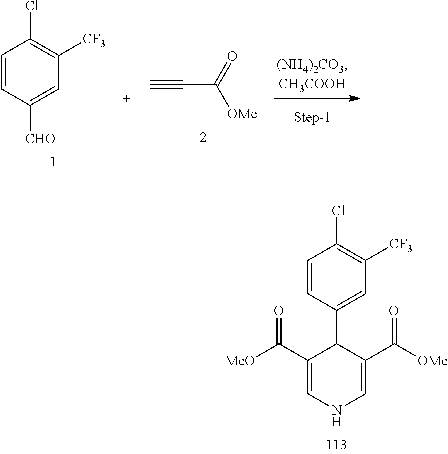

To a stirred solution of 4-chloro-3-(trifluoromethyl)benzaldehyde (1, 0.100 g, 0.4807 mmol) and methyl propiolate (2, 0.080 g, 0.9615 mmol) in acetic acid (10 mL), (NH₄)₂CO₃ (0.046 g, 0.4807) was added slowly at RT and the reaction mixture was heated at 70° C. for 14 h. After complete consumption of the starting material (monitored by TLC), the reaction mixture was cooled to RT and slowly quenched with ice cold water (10 mL). The aqueous layer was extracted with DCM (20 mL×2). The combined organic layers were washed with brine (10 mL), dried over Na₂SO₄ and concentrated under reduced pressure to obtain the crude product. The crude product was purified by combi-flash column chromatography using 0-40% EtOAc in n-hexane to afford 113 as a pale yellow solid (0.040 g, 22%). TLC: 40% EtOAc/hexane (R_{f}: 0.8).

¹H NMR (400 MHz, DMSO-d₆) δ 9.33 (br. s, 1H), 7.63 (d, J=8.01 Hz, 1H), 7.57 (s, 1H), 7.40-7.50 (m, 3H), 4.79 (s, 1H), 3.35 (s, 6H).

LC-MS: 99.75%, m/z=376.0 (M+H)⁺ (Column: X-Select CSH C18 (3.0×50 mm, 2.5 μm); Rₜ: 1.986 min; A: 0.05% formic acid in water: ACN (95:05), B: 0.05% formic acid in ACN; Temperature: 50° C.; and Flow Rate: 1.2 mL/min).

HPLC: 99.42% (Column: X-SELECT CSH C-18 (4.6×150 mm, 3.5 μm); Rₜ: 8.715 min, A-0.1% formic acid in water, B: ACN T/B %: 0.01/5, 1.0/5, 8.0/100, 12.0/100, 14.0/5, 18.0/5; and Flow Rate: 1.0 mL/min).

### Example 2

The panel of dihydropyridines on the K_{Ca}3.1 channel was screened using patch-clamp to understand the structure-activity relationships.

K_{Ca}3.1 Patch-Clamp Protocol

The effect of the compounds on K_{Ca}3.1 channel was evaluated by patch-clamp using a QPatch HTX automated electrophysiology platform (Sophion, Denmark) and by manual patch-clamp (HEKA, Germany). For QPatch experiments, the giga-seal and whole-cell requirements for the automated electrophysiology were the following: minimum seal resistance=0.1 GQ; holding potential=−80 mV; holding pressure=−20 mbar; and positioning pressure=−70 mbar. For K_{Ca}3.1 experiment, the external solution was Na⁺-Ringer's and contained (in mM): 160 NaCl; 10 HEPES; 4.5 KCl; 1 MgCl₂; and 2 CaCl₂ (pH=7.4, 310 mOsm). The internal solution contained (in mM): 120 KCl; 10 HEPES; 1.75 MgCl₂; 1 Na₂ATP; 10 EGTA; and 8.6 CaCl₂ (1 μM free Ca²⁺) (pH=7.2, 300 mOsm). Following establishment of the whole-cell configuration, cells were held at −80 mV and K_{Ca}3.1 currents elicited by a voltage protocol that held at −80 mV for 20 ms, stepped to −120 mV for 20 ms, ramped from −120 to +40 mV over 200 ms, and then stepped back to −120 mV for 20 ms. This pulse protocol was applied every 10 s. Current slopes (in amperes per second) were measured using the Sophion QPatch software and exported to Microsoft Excel and GraphPad Prism 7 for analysis. The decreases in the slope between −85 and −65 mV were used to determine the IC₅₀ for K_{Ca}3.1 inhibition. Curve-fitting and IC₅₀ determination were performed using GraphPad Prism software.

Results and Discussion

The IC₅₀ of the dihydropyridines are summarised in Table 2. The first group blocked K_{Ca}3.1 with IC₅₀ values greater than 5 μM. The second group blocked the channel at 1-5 μM. The third group blocked K_{Ca}3.1 at 100-200 nM. The fourth group blocked K_{Ca}3.1 at low nanomolar concentrations. The effects of selected group 2, 3, 4a, 4b and 4c compounds on K_{Ca}3.1 currents are shown in FIG. 3.

TABLE 2
Structure-activity-relationships. Four groups of inhibitors, their chemical
substitutions and potencies for block of K_{Ca}3.1 are shown.

| | | | | | | | | | |
|---|---|---|---|---|---|---|---|---|---|
| | | | | | | | | | IC₅₀ |
| Name | R1 | R2 | R3 | R4 | R5 | R6 | R7 | R8 | (nM) |

Group 1

| | | | | | | | | | |
|---|---|---|---|---|---|---|---|---|---|
| 01 | — | — | — | Cl | methyl | methyl | CH₃ | 2- | >10000 |
| | | | | | acetate | acetate | | ethoxy- | |
| | | | | | | | | ethan- | |
| | | | | | | | | 1-amine | |
| 02 | — | — | — | Cl | methyl | ethyl | CH₃ | 2- | >10000 |
| | | | | | acetate | acetate | | ethoxy- | |
| | | | | | | | | ethan- | |
| | | | | | | | | 1-amine | |
| 03 | — | — | NO₂ | — | methyl | 2-(4- | CH₃ | CH₃ | >10000 |
| | | | | | acetate | benz- | | | |
| | | | | | | hydryl- | | | |
| | | | | | | piperazin- | | | |
| | | | | | | 1-yl)ethyl | | | |
| | | | | | | acetate | | | |
| 04 | — | — | NO₂ | — | iso- | 2-meth- | CH₃ | CH₃ | >10000 |
| | | | | | propyl | oxyethyl | | | |
| | | | | | acetate | acetate | | | |
| 05 | — | — | NO₂ | — | iso- | methyl | CH₃ | N | 5000 |
| | | | | | propyl | acetate | | | |
| | | | | | acetate | | | | |
| 06 | — | — | NO₂ | — | 2- | 2- | CH₃ | CH₃ | 9880 |
| | | | | | meth- | meth- | | | |
| | | | | | oxyethyl | oxyethyl | | | |
| | | | | | acetate | acetate | | | |
| 72 | — | — | NO₂ | — | methyl | cyclopent- | CH₃ | CH₃ | >10000 |
| | | | | | acetate | 2-en- | | | |
| | | | | | | 1-one | | | |
| 77 | — | — | NO₂ | — | methyl | 3,4- | CH₃ | H | >10000 |
| | | | | | acetate | dihydro- | | | |
| | | | | | | 2H- | | | |
| | | | | | | thiopyran | | | |
| | | | | | | 1,1- | | | |
| | | | | | | dioxide | | | |
| 76 | — | — | NO₂ | — | methyl | 2,3- | CH₃ | CH₃ | 5000 |
| | | | | | acetate | dihydro- | | | |
| | | | | | | thiophene | | | |
| | | | | | | 1,1- | | | |
| | | | | | | dioxide | | | |
| 87 | — | — | NO₂ | — | cyclo- | cyclo- | CH₃ | H | 6000 |
| | | | | | hex- | hex- | | | |
| | | | | | 2-en- | 2-en- | | | |
| | | | | | 1-one | 1-one | | | |

Group 2

| | | | | | | | | | |
|---|---|---|---|---|---|---|---|---|---|
| 07 | — | — | — | NO₂ | methyl | methyl | CH₃ | CH₃ | 4000 |
| | | | | | acetate | acetate | | | |
| 73 | — | — | NO₂ | — | methyl | cyclohex- | CH₃ | H | 1920 |
| | | | | | acetate | 2-en-1-one | | | |
| 75 | — | — | NO₂ | — | methyl | (methyl- | CH₃ | CH₃ | 2800 |
| | | | | | acetate | sulfonyl) | | | |
| | | | | | | methane | | | |
| 08 | — | — | NO₂ | — | methyl | ethyl | CH₃ | CH₃ | 2300 |
| | | | | | acetate | acetate | | | |
| 56 | — | — | — | — | methyl | propan- | CH₃ | CH₃ | 1795 |
| | | | | | acetate | 2-one | | | |
| 85 | — | — | NO₂ | — | propan- | propan- | CH₃ | CH₃ | 1102 |
| | | | | | 2-one | 2-one | | | |
| 105 | F | methyl | CF₃ | — | methyl | methyl | CH₃ | CH₃ | >1000 |
| | | acetate | | | acetate | acetate | | | |
| 106 | F | Aceta- | CF₃ | — | methyl | methyl | CH₃ | CH₃ | >1000 |
| | | mide | | | acetate | acetate | | | |

Group 3

| | | | | | | | | | |
|---|---|---|---|---|---|---|---|---|---|
| 79 | — | — | NO₂ | — | methyl | propan- | CH₃ | CH₃ | 142 ± 7.6 |
| | | | | | acetate | 2-one | | | |
| 84 | — | — | NO₂ | — | methyl | methyl | CH₃ | CH₃ | 102 ± 11 |
| | | | | | acetate | acetate | | | |

Group 4a*

| | | | | | | | | | |
|---|---|---|---|---|---|---|---|---|---|
| 57 | F | — | CF₃ | — | methyl | propan- | CH₃ | CH₃ | 16.8 ± 0.9 |
| | | | | | acetate | 2-one | | | |
| 101 | F | — | CF₃ | — | methyl | methyl | CH₃ | CH₃ | 16 ± 0.2 |
| | | | | | acetate | acetate | | | |
| 102 | Cl | — | CF₃ | — | methyl | methyl | CH₃ | CH₃ | 7 ± 0.2 |
| | | | | | acetate | acetate | | | |

Group 4b*

| | | | | | | | | | |
|---|---|---|---|---|---|---|---|---|---|
| 104 | F | — | CF₃ | — | methyl | methyl | H | CH₃ | 7.6 ± 1.7 |
| | | | | | acetate | acetate | | | |

Group 4c*

| | | | | | | | | | |
|---|---|---|---|---|---|---|---|---|---|
| 103 | F | — | CF₃ | — | methyl | methyl | H | H | 6.1 ± 0.9 |
| | | | | | acetate | acetate | | | |
| 108 | H | — | CF₃ | — | methyl | methyl | H | H | 18.5 ± 1.1 |
| | | | | | acetate | acetate | | | |
| 109 | H | F | CF₃ | | methyl | methyl | H | H | 4.5 ± 0.2 |
| | | | | | acetate | acetate | | | |
| 110 | F | F | CF₃ | | methyl | methyl | H | H | 6.9 ± 1.3 |
| | | | | | acetate | acetate | | | |
| 113 | Cl | — | CF₃ | | methyl | methyl | H | H | 5.7 ± 0.3 |
| | | | | | acetate | acetate | | | |

*IC₅₀ values determined by automated planar patch clamp for Group-4 compounds were slightly higher than IC₅₀ values determined by manual patch.

### Example 3

Since dihydropyridines inhibit L-type voltage-gated calcium channels, selected analogues were tested on Ca_{V}1.2.

Ca_{V}1.2 Patch-Clamp Protocol

The effect of the compounds on Ca_{V}1.2 channel was evaluated by manual patch-clamp with an EPC-10 HEKA amplifier. The external solution contained (in mM): 140 TEA-Cl; 2 MgCl₂; 10 CaCl₂; 10 HEPES; and 5 D-glucose (pH=7.4). The internal solution contained (in mM): 120 CsCl; 1 MgCl₂; 10 HEPES; 10 EGTA; 0.3 Na₂-GTP; and 4 Mg-ATP (pH=7.2). Following establishment of the whole-cell configuration, Ca_{V}1.2 current was recorded at holding membrane potential of −80 mV and then depolarized to +10 mV (test pulse was modified slightly due to lead IV test) for 0.3 s. This protocol was repeated at 20 s intervals to observe the effect of the test compounds on the peak of Ca_{V}1.2 current. Each cell was incubated with each test compound for 5 min, or until the current reached steady-state. The compounds were applied at multiple concentrations from low to high. Each cell acted as its own control. The decreases in the peak currents were used to determine the IC₅₀ for Ca_{V}1.2 inhibition. Curve-fitting and IC₅₀ calculations were performed using IGOR software.

Results and Discussion

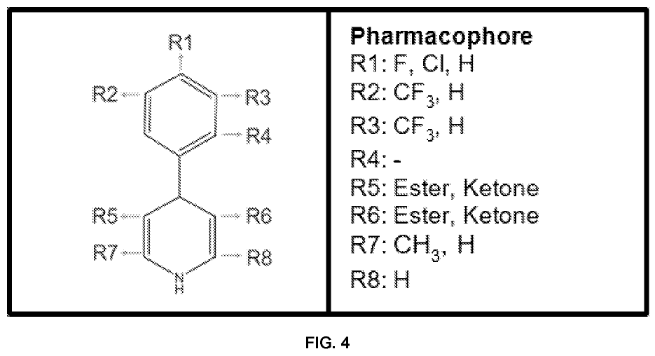

Table 3 shows the selected analogues and compares the selectivity of the selected analogues for K_{Ca}3.1 and Ca_{V}1.2. Group 2 analogues showed selectivity for Ca_{V}1.2 over K_{Ca}3.1. Group 3 analogues showed ˜10-fold selectivity for K_{Ca}3.1 over Ca_{V}1.2. Group 4 analogues showed >50-fold selectivity for K_{Ca}3.1 over Ca_{V}1.2, with group 4c analogues being the most selective for K_{Ca}3.1. The pharmacophore required for low nM IC₅₀ block of K_{Ca}3.1, and >1000-fold selectivity for K_{Ca}3.1 over Ca_{V}1.2 is shown in FIG. 4.

TABLE 3
Selectivity of compounds for K_{Ca}3.1 over Ca_{V}1.2.

| | Group | | | | | | | | | | | |
|---|---|---|---|---|---|---|---|---|---|---|---|---|
| | Group 2 | | Group 3 | Group 4a | | | Group 4b | Group 4c | | | | |
| Compound | 07 | 08 | 79 | 57 | 101 | 102 | 104 | 103 | 108 | 109 | 110 | 113 |
| R7 | CH₃ | CH₃ | CH₃ | CH₃ | CH₃ | CH₃ | H | H | H | H | H | H |
| R8 | CH₃ | CH₃ | CH₃ | CH₃ | CH₃ | CH₃ | CH₃ | H | H | H | H | H |
| K_{Ca}3.1 | 4,000 | 2,300 | 142 | 16.8 | 16 | 7 | 7.6 | 6.1 | 18.5 | 4.5 | 6.9 | 5.7 |
| IC₅₀ (nM) | | | | | | | | | | | | |
| Ca_{V}1.2 | 16^{#} | 0.38^{#} | 2000 | 1500 | 3200 | 2400 | 4900 | 13500 | 22000 | 10600 | 9100 | 3700 |
| IC₅₀ (nM) | | | | | | | | | | | | |
| Ca_{V}1.2/K_{Ca}3.1 | 0.004 | 0.0001 | 14 | 89 | 200 | 342 | 644 | 2213 | 1189 | 2355 | 1318 | 649 |
| selectivity ratio | | | | | | | | | | | | |

^{#}reported values (Y. Kuryshev et. al., Assay Drug Dev Technol. 2014, 12(2), 110-119; Eurofins Panlab)

### Example 4

Selected analogues from Group 4 were tested on related potassium channels.

Potassium Channels Patch-Clamp Protocol

The effects of Group 4 compounds on related potassium channels were evaluated by patch-clamp using a QPatch HTX automated electrophysiology platform (Sophion, Denmark). The giga-seal and whole-cell requirements for the automated electrophysiology were the following: minimum seal resistance=0.1 GO, holding potential=−80 mV, holding pressure=−20 mbar; and positioning pressure=−70 mbar. Buffer compositions and voltage protocols for each specific ion channels tested are shown in Table 4.

TABLE 4
Buffer compositions and voltage protocols for potassium channels tested.

| | | External buffer | Internal buffer | |
|---|---|---|---|---|
| Channel | Cell line | (mM) | (mM) | Pulse protocol |
| K_{Ca}1.1 | HEK | 4.5 KCl, 160 | 160 KF, 2 MgCl₂, | 30 ms depolarizing pulses to +160 mV were applied |
| | Human | NaCl, 1 MgCl₂, 2 | 10 EGTA, 10 | every 10 s. The amplitudes of peak current were |
| | K_{Ca}1.1² | CaCl₂, 10 | HEPES; pH 7.2, | measured using the Sophion QPatch software and |
| | | HEPES; pH 7.2, | 290-320 mOsm. | exported to Microsoft Excel and GraphPad Prism 7 for |
| | | 290-320 mOsm | | analysis. |
| K_{Ca}2.2 | HEK | 160 NaCl, 10 | 120 KCl, 10 | Holding at −80 mV for 20 ms, −120 mV for 20 ms, |
| | Rat K_{Ca}2.2 | HEPES, 4.5 KCl, | HEPES, 1.75 | ramped from −120 to +40 mV over 200 ms, and then |
| | | 1 MgCl₂, 2 | MgCl₂, 4 Na₂ATP, | stepped back to −120 mV for 20 ms. Current slopes (in |
| | | CaCl₂, 10 | 10 EGTA, 8.6 | amperes per second) were measured using the |
| | | Glucose, (pH | CaCl₂ (1 μM free | Sophion QPatch software and exported to Microsoft |
| | | 7.4, 310 mOsm) | Ca²⁺) (pH 7.2, 300 | Excel and GraphPad Prism 7 for analysis. Decreases |
| | | | mOsm) | in slope between −85 and −65 mV was used to |
| | | | | determine the inhibition. |
| K_{Ca}2.3 | COS7 | 160 NaCl, 10 | 120 KCl, 10 | Holding at −80 mV for 20 ms, −120 mV for 20 ms, |
| | Human | HEPES, 4.5 KCl, | HEPES, 1.75 | ramped from −120 to +40 mV over 200 ms, and then |
| | K_{Ca}2.3 | 1 MgCl₂, 2 | MgCl₂, 4 Na₂ATP, | stepped back to −120 mV for 20 ms. Current slopes (in |
| | | CaCl₂, 10 | 10 EGTA, 8.6 | amperes per second) were measured using the |
| | | Glucose, (pH | CaCl₂ (1 μM free | Sophion QPatch software and exported to Microsoft |
| | | 7.4, 310 mOsm) | Ca²⁺) (pH 7.2, 300 | Excel and GraphPad Prism 7 for analysis. Decreases |
| | | | mOsm) | in slope between −85 and −65 mV was used to |
| | | | | determine the inhibition. |
| K_{V}1.1 | L929 | 4.5 KCl, 160 | 160 KF, 2 MgCl₂, | 200 ms depolarizing pulses to +40 mV from the |
| | Mouse | NaCl, 1 MgCl₂, 2 | 10 EGTA, 10 | holding potential of −80 mV applied every 10 s. The |
| | K_{V}1.1³ | CaCl₂, 10 | HEPES; pH 7.2, | amplitudes of peak current were measured using the |
| | | HEPES; pH 7.2, | 290-320 mOsm | Sophion QPatch software and exported to Microsoft |
| | | 290-320 mOsm | | Excel and GraphPad Prism 7 for analysis. |
| K_{V}1.2 | B82 | 4.5 KCl, 160 | 160 KF, 2 MgCl₂, | 200 ms depolarizing pulses to +40 mV from the |
| | Rat K_{V}1.2³ | NaCl, 1 MgCl₂, 2 | 10 EGTA, 10 | holding potential of −80 mV applied every 30 s. The |
| | | CaCl₂, 10 | HEPES; pH 7.2, | amplitudes of peak current were measured using the |
| | | HEPES; pH 7.2, | 290-320 mOsm | Sophion QPatch software and exported to Microsoft |
| | | 290-320 mOsm | | Excel and GraphPad Prism 7 for analysis. |
| K_{V}1.3 | L929 | 4.5 KCl, 160 | 160 KF, 2 MgCl₂, | 200 ms depolarizing pulses to +40 mV from the |
| | Mouse | NaCl, 1 MgCl₂, 2 | 10 EGTA, 10 | holding potential of −80 mV applied every 45 s. The |
| | K_{V}1.3³ | CaCl₂, 10 | HEPES; pH 7.2, | amplitudes of peak current were measured using the |
| | | HEPES; pH 7.2, | 290-320 mOsm | Sophion QPatch software and exported to Microsoft |
| | | 290-320 mOsm | | Excel and GraphPad Prism 7 for analysis. |
| K_{V}1.4 | LTK | 4.5 KCl, 160 | 160 KF, 2 MgCl₂, | 200 ms depolarizing pulses to +40 mV from the |
| | Human | NaCl, 1 MgCl₂, 2 | 10 EGTA, 10 | holding potential of −80 mV applied every 30 s. The |
| | K_{V}1.4 | CaCl₂, 10 | HEPES; pH 7.2, | amplitudes of peak current were measured using the |
| | | HEPES; pH 7.2, | 290-320 mOsm | Sophion QPatch software and exported to Microsoft |
| | | 290-320 mOsm | | Excel and GraphPad Prism 7 for analysis. |
| K_{V}1.5 | MEL | 4.5 KCl, 160 | 160 KF, 2 MgCl₂, | 200 ms depolarizing pulses to +60 mV from the |
| | Human | NaCl, 1 MgCl₂, 2 | 10 EGTA, 10 | holding potential of −80 mV applied every 30 s. The |
| | K_{V}1.5³ | CaCl₂, 10 | HEPES; pH 7.2, | amplitudes of peak current were measured using the |
| | | HEPES; pH 7.2, | 290-320 mOsm. | Sophion QPatch software and exported to Microsoft |
| | | 290-320 mOsm | | Excel and GraphPad Prism 7 for analysis. |
| K_{V}1.7 | CHL | 4.5 KCl, 160 | 160 KF, 2 MgCl₂, | 200 ms depolarizing pulses to +40 mV from the |
| | Human | NaCl, 1 MgCl₂, 2 | 10 EGTA, 10 | holding potential of −80 mV applied every 30 s. The |
| | K_{V}1.7 | CaCl₂, 10 | HEPES; pH 7.2, | amplitudes of peak current were measured using the |
| | | HEPES; pH 7.2, | 290-320 mOsm. | Sophion QPatch software and exported to Microsoft |
| | | 290-320 mOsm | | Excel and GraphPad Prism 7 for analysis. |
| K_{V}3.1 | L929 | 4.5 KCl, 160 | 160 KF, 2 MgCl₂, | 200 ms depolarizing pulses to +40 mV from the |
| | Mouse | NaCl, 1 MgCl₂, 2 | 10 EGTA, 10 | holding potential of −90 mV applied every 45 s. The |
| | K_{V}3.1³ | CaCl₂, 10 | HEPES; pH 7.2, | amplitudes of peak current were measured using the |
| | | HEPES; pH 7.2, | 290-320 mOsm. | Sophion QPatch software and exported to Microsoft |
| | | 290-320 mOsm | | Excel and GraphPad Prism 7 for analysis. |
| K_{V}4.2 | LTK | 4.5 KCl, 160 | 160 KF, 2 MgCl₂, | 200 ms depolarizing pulses to +40 mV from the |
| | Rat K_{V}4.2 | NaCl, 1 MgCl₂, 2 | 10 EGTA, 10 | holding potential of −80 mV applied every 15 s. The |
| | | CaCl₂, 10 | HEPES; pH 7.2, | amplitudes of peak current were measured using the |
| | | HEPES; pH 7.2, | 290-320 mOsm. | Sophion QPatch software and exported to Microsoft |
| | | 290-320 mOsm | | Excel and GraphPad Prism 7 for analysis. |
| K_{V}11.1 | CHO | 145 NaCl, 10 | 120 KCl, 10 | Cells were held at −80 mV. Every 15 s, a 80 ms |
| | Human | HEPES, 4 KCl, 1 | HEPES, 1.75 | voltage step to −50 mV was applied and then a 4.8 s |
| | K_{V}11.1, | MgCl₂, 2 CaCl₂, | MgCl₂, 2 Na₂ATP, | depolarizing voltage step from −50 mV to +30 mV was |
| | B'SYS | 10 Glucose, pH | 10 EGTA, 5.4 | followed by a 5 s tail step to −50 mV, then step back |
| | GmbH, | 7.4, 305 mOsm | CaCl₂ (PH 7.2, | to −80 mV for 3.1 s. The amplitude of the peak current |
| | Switzerland | | 295 mOsm) | at the beginning of the tail step was measured by the |
| | | | | Sophion QPatch software and exported to Microsoft |
| | | | | Excel and GraphPad Prism 7 for analysis. |

Results and Discussion

Group 4a, 4b and 4c analogues exhibited >1000-fold selectivity for K_{Ca}3.1 over related potassium channels (Table 5).

TABLE 5
Selectivity of Group 4 compounds for K_{Ca}3.1 over related K⁺ channels.

| | Group 4a | Group 4b | Group 4c | | | | |
|---|---|---|---|---|---|---|---|
| Compound | 101 | 104 | 103 | 108 | 109 | 110 | 113 |
| K_{Ca}3.1 | 16.2 ± 0.2 nM | 7.6 ± 1.7nM | 6.1 ± 0.9 nM | 18.5 ± 1.1 nM | 4.5 ± 0.2 nM | 6.9 ± 1.3 nM | 5.7 ± 0.3 nM |
| K_{Ca}1.1 | >10 μM | >10 μM | >10 μM | >10 μM | not tested | not tested | not tested |
| K_{Ca}2.2 | >10 μM | >10 μM | >10 μM | >10 μM | not tested | not tested | not tested |
| K_{Ca}2.3 | 8 μM | 8 μM | 6 μM | 7 μM | >10 μM | >10 μM | >10 μM |
| K_{V}1.1 | >10 μM | >10 μM | >10 μM | >10 μM | not tested | not tested | not tested |
| K_{V}1.2 | >10 μM | >10 μM | >10 μM | >10 μM | not tested | not tested | not tested |
| K_{V}1.3 | >10 μM | >10 μM | >10 μM | >10 μM | >10 μM | >10 μM | >10 μM |
| K_{V}1.4 | >10 μM | >10 μM | >10 μM | >10 μM | not tested | not tested | not tested |
| K_{V}1.5 | >10 μM | >10 μM | >10 μM | >10 μM | >10 μM | >10 μM | >10 μM |
| K_{V}1.7 | >10 μM | >10 μM | >10 μM | >10 μM | not tested | not tested | not tested |
| K_{V}3.1 | >10 μM | >10 μM | >10 μM | >10 μM | not tested | not tested | not tested |
| K_{V}4.2 | >10 μM | >10 μM | >10 μM | >10 μM | not tested | not tested | not tested |
| K_{V}11.1 | >10 μM | >10 μM | >10 μM | >10 μM | >10 μM | >10 μM | >10 μM |
| (hERG) | | | | | | | |

### Example 5

Compounds 101 and 103 from Group 4 were tested for their ability to inhibit cytochrome P450 enzymes. In addition, their plasma protein binding and microsomal stability were evaluated.

Cytochrome P450 Enzymes Inhibition Assay

Microsomal suspension in potassium phosphate buffer (50 mM, pH=7.4) was mixed with the respective probe substrates before equilibration at 37° C. for 5 min. DMSO control, reference inhibitors (Ketoconazole for 3A4, Quinidine for 2D6, Sulphaphenazolefor for 2C9, Nootkatone for 2C19 and Furafylline for 1A2) and compound 101 or 103 at various concentrations (0.009 μM, 0.027 μM, 0.082 μM, 0.247 μM, 0.741 μM, 2.222 μM, 6.667 μM and 20 μM) were spiked to the prepared microsomal and probe substrate mixtures and incubated in a shaking water bath at 37° C. for 5 min. NADPH (10 mM) was added to all the samples to initiate the reaction. ACN containing verapamil (200 ng) and warfarin (200 ng) as internal standards was added to the mixture to stop the reaction after 10 min of incubation at 37° C. The samples were vortexed gently and centrifuged at 1021 g for 20 min at 4° C. before they were injected into the LC-MS/MS system. The metabolite formation for each substrate under the above conditions was estimated using the following formula: Percent inhibition=[1−(test area ratio/control area ratio)]×100. The IC₅₀ values were determined using a sigmoidal dose-response curve (variable slope) in GraphPad Prism® 5 software.

Plasma Protein Binding Assay

Human and mouse plasma (150 μL) containing test compound (3 μM, final concentration) and sodium phosphate buffer (150 μL, 100 mM, pH 7.4) were added to the equilibrium dialysis device and equilibrated at 37° C. for 4.5 h with constant shaking. After equilibration, 10 μL of the plasma was taken out from the first half of the well and mixed with 50 μL of blank buffer, and later quenched with 200 μL of 0.05% formic acid in ACN containing 100 ng/mL of Loperamide, Phenacetin & Tolbutamide as internal standards. Similarly, 50 μL of plasma was taken from the second half of the well and mixed with 10 μL of blank plasma before quenching. To prepare the recovery sample, 10 μL of plasma containing 3 μM of compound 101 or 103 was added to 50 μL of blank buffer and quenched. All samples were centrifuged at 14,000 rpm for 5 min at 4° C. Warfarin and naltrexone were used as positive controls. Supernatant of all samples were analyzed by LC-MS/MS.

Microsomal Stability Studies

Human microsomes (20 mg/mL) were suspended in potassium phosphate buffer (66.7 mM, pH=7.4) as the incubation mixture. 1 μL of the test compound or control (1.1 mM) was added to the incubation mixture to give a working concentration of 1.1 μM, and aliquoted into 4 tubes for 4 time point assays (0, 5, 15 and 30 min). All the tubes and NADPH solution (10 mM) were preincubated at 37° C. for 5 min in a shaking water bath. After preincubation, NADPH solutions (10 mM) was added to the 5-, 15- and 30-min tubes. Buffer was added to the 0-min tube to obtain a final concentration of 1 μM for the test compounds or reference standard. At the end of the incubation period for each respective tube, quenching solution (ACN containing 100 ng/mL of Warfarin and Loperamide as internal standards) was added to stop the reaction. Resulting samples were centrifuged at 3,220 g for 20 min and supernatant from each reaction tube was taken for LC-MS/MS analysis.

Results and Discussion

Group 4c analogues were more selective for K_{Ca}3.1 over cytochrome P450 enzymes, exhibited less plasma protein binding, lower c Log P values, and greater stability and solubility than Group 4a compounds (Table 6).

TABLE 6
Comparison of Group 4a versus Group 4c analogues.

| | Group 4a | Group 4c |
|---|---|---|
| Compound | 101 | 103 |

Inhibition of cytochrome P450 enzymes

| CYP-3A4 | IC₅₀ uM | 1.3 | μM | ~20 | μM |
|---|---|---|---|---|---|
| | CYP IC₅₀/K_{Ca}3.1 IC₅₀ | 79.75 | | 3279 | |
| CYP-2C9 | IC₅₀ uM | 2.6 | μM | 5.1 | μM |
| | CYP IC₅₀/K_{Ca}3.1 IC₅₀ | 162.5 | | 836 | |
| CYP-IA2 | IC₅₀ uM | 9.1 | μM | >20 | μM |
| | CYP IC₅₀/K_{Ca}3.1 IC₅₀ | 558 | | 3279 | |
| CYP-2D6 | IC₅₀ uM | >20 | μM | >20 | μM |
| | CYP IC₅₀/K_{Ca}3.1 IC₅₀ | 1226 | | 3279 | |
| CYP-2C19 | IC₅₀ uM | >20 | μM | 20 | μM |

| | | |
|---|---|---|
| CYP IC₅₀/K_{Ca}3.1 IC₅₀ | 1226 | 3279 |

Plasma Protein Binding

| | | |
|---|---|---|
| Human | 99.27% | 97.76% |
| Mouse | 99.12% | 94.18% |

Microsomal Stability (human)

| | | |
|---|---|---|
| % remaining @ 30 mins | 53% (control: Verapamil, 33%) | 25% (Verapamil, 7%) |
| drug/control | 1.6 | 3.7 |
| T_{1/2} | 22 min (Verapamil, 20 min) | 16 min (Verapamil, 8 min) |
| drug/control | 1.1 | 2.0 |

cLogP

| | |
|---|---|
| 4.49 | 3.45 |

Since compound 103 from Group 4c blocks K_{Ca}3.1 with low nanomolar potency (IC₅₀=6 nM) and has >1000-fold selectivity for K_{Ca}3.1 over other channels and a panel of ˜100 molecular targets, it was used in the subsequent studies.

### Example 6

The selectivity of compound 103 was assessed by competitive binding, enzyme and uptake assays against 97 targets.

Competitive Binding Assay

The experimental conditions for the individual competitive binding assay are shown in Table 7. Compound 103 was tested at 3 μM. The results are expressed as a percent inhibition of control specific binding [100-(measured specific binding/control specific binding*100)]. The IC₅₀ values and Hill coefficients (nH) were determined by non-linear regression analysis of the competition curves generated with mean replicate values using Hill equation curve fitting performed with software developed at Cerep (Hill software) and validated by comparison with data generated by the commercial software SigmaPlot® 4.0.

TABLE 7
Experimental conditions for competitive binding assay.

| | | | | | | | |
|---|---|---|---|---|---|---|---|
| | | | | | | | Detection |
| Assay | Source | Ligand | Conc. | Kd | Non Specific | Incubation | Method |

[symbol]

[symbol]

[symbol]

indicates data missing or illegible when filed

Enzyme and Uptake Assay

Experimental conditions for the individual enzyme and uptake assay are shown in the table below. Compound 103 was tested at 3 μM. The results are expressed as a percent inhibition of control specific activity [100−(measured specific activity/control specific activity*100)]. The IC₅₀ values, EC₅₀ values and Hill coefficients (nH) were determined by non-linear regression analysis of the inhibition/concentration-response curves generated with mean replicate values using Hill equation curve fitting performed with software developed at Cerep (Hill software) and validated by comparison with data generated by the commercial software SigmaPlot® 4.0.

TABLE 8
Experimental conditions for the individual enzyme and uptake assay.

| | | | | | |
|---|---|---|---|---|---|
| | | | | Measured | Detection |
| Assay | Source | [symbol] /Stimulus/ [symbol] | Incubation | Component | Method |

[symbol]

| | | | | | |
|---|---|---|---|---|---|
| PKCα (h) | human | ATP + CRE [symbol] | 15 min | phospho-CR [symbol] | LANCE |
| | [symbol] | (CKRREILSRRPSYRK) | RT | (CKRREILSRRPSYRK) | |
| | (insect cells) | (20 nM) | | | |

Epigenetic enzymes and DNA related enzymes

| | | | | | |
|---|---|---|---|---|---|
| [symbol] (h) | human | ATP | 5 min | inorganic phosphate | [symbol] |
| | recombinant | (100 μM) | RT | | |
| | (E. coli) | | | | |
| [symbol] (h) | human | ATP | 10 min | inorganic phosphate | [symbol] |
| | recombinant | (50 μM) | RT | | |
| HDAC3 (h) | human | [symbol] HDAC [symbol] | 15 min | [symbol] | [symbol] |
| | [symbol] | (50 μM) | RT | | |
| HDAC4 (h) | human | [symbol] HDAC [symbol] | 30 min | [symbol] | [symbol] |
| | recombinant | (20 μM) | RT | | |
| HDAC8 (h) | human | [symbol] HDAC [symbol] | [symbol] 0 min | [symbol] | [symbol] |
| | recombinant | (25 μM) | RT | | |
| HDAC11 (h) | human | [symbol] HDAC [symbol] | [symbol] 0 min | [symbol] | [symbol] |
| | recombinant | (50 μM) | 37° C. | | |
| [symbol] 1 (h) | human | [symbol] HDAC [symbol] | 20 min | [symbol] | [symbol] |
| (inhibitor effect) | recombinant | (200 μM) | RT | | |
| | (E. coli) | | | | |
| [symbol] 2 (h) | human | [symbol] 2 [symbol] | 60 min | [symbol] | [symbol] |
| (inhibitor effect) | recombinant | [symbol] | RT | | |
| | (E. coli) | (150 μM) | | | |

Other enzymes

| | | | | | |
|---|---|---|---|---|---|
| COX1(h) | human | [symbol] acid (2 μM) + | [symbol] min | [symbol] | [symbol] |
| | recombinant | ADHP (25 μM) | RT | ( [symbol] ADHP) | |
| 5-lipoxygenase (h) | human | [symbol] acid | 5 min | [symbol] 12 [symbol] | [symbol] |
| | recombinant | (2 [symbol] μM) | RT | | |
| | ( [symbol] cells) | | | | |
| | ( [symbol] ) | | | | |
| [symbol] (h) | human | [ [symbol] ] [symbol] + [symbol] | 20 min | [ [symbol] ] [symbol] GMP | [symbol] |
| | recombinant | (1. [symbol] μM) | RT | | |
| | ( [symbol] cells) | | | | |
| [symbol] E2A1 (h) | human | [ [symbol] ]cAMP + cAMP | 15 min | [ [symbol] ] [symbol] AMP | [symbol] |
| | recombinant | (2 μM) | RT | | |
| | ( [symbol] cells) | | | | |
| [symbol] E3A (h) | human | [ [symbol] ]cAMP + cAMP | 15 min | [ [symbol] ] [symbol] AMP | [symbol] |
| | recombinant | (0.5 [symbol] M) | RT | | |
| | ( [symbol] cells) | | | | |
| [symbol] 2 (h) | human | [ [symbol] ]cAMP + cAMP | 20 min | [ [symbol] ] [symbol] AMP | [symbol] |
| | recombinant | (0. [symbol] μM) | RT | | |
| | ( [symbol] cells) | | | | |
| [symbol] (h) | human | [ [symbol] ] [symbol] + [symbol] | [symbol] 0 min | [ [symbol] ] [symbol] GMP | [symbol] |
| (activator effect) | platelets | (1 μM) | RT | | |
| [symbol] | CHO cells | [symbol] | 10 min | cAMP | HTRF |
| (activator effect) | | ( [symbol] μM [symbol] for [symbol] ) | RT | | |
| [symbol] (h) | human | GTP | 10 min | [symbol] | HTRF |
| (activator effect) | recombinant | (10 μM) | RT | | |
| | | (100 μM [symbol] for [symbol] ) | | | |
| phosphatase 18 (h) | human | [symbol] | 30 min | [symbol] | [symbol] |
| (PIP1B) | recombinant | (10 μM) | RT | | |
| | (E. coli) | | | | |
| phosphatase | human | [symbol] | 20 min | [symbol] | [symbol] |
| CDC25A (h) | recombinant | (200 μM) | RT | | |
| | (E. coli) | | | | |
| acetylcholinesterase | human | [symbol] | 30 min | [symbol] 2 [symbol] acid | [symbol] |
| (h) | recombinant | ( [symbol] μM) | RT | | |
| | (HEX- [symbol] cells) | | | | |
| GABA [symbol] | [symbol] | GABA (9 mM) + | 60 min | [symbol] | [symbol] |
| | | [symbol] (9 mM) | 37° C. | | |
| MAO-A (h) | human | [symbol] | 20 min | 4- [symbol] | [symbol] |
| | platelets | (0.15 [symbol] M) | RT | | |
| MAO-B (h) | human | [symbol] | 60 min | methyl ester [symbol] | [symbol] |
| recombinant enzyme | recombinant | (4 μM) | 37° C. | | |
| tyrosine hydroxylase | [symbol] | [ [symbol] ] [symbol] | 20 min | [symbol] | [symbol] |
| | | (10 μM) | 37° C. | | |
| ATPase ( [symbol] ) | [symbol] | [symbol] | [symbol] 0 min | [symbol] | [symbol] |
| | | (2 nM) | 37° C. | | |

[symbol]

indicates data missing or illegible when filed

Results and Discussion

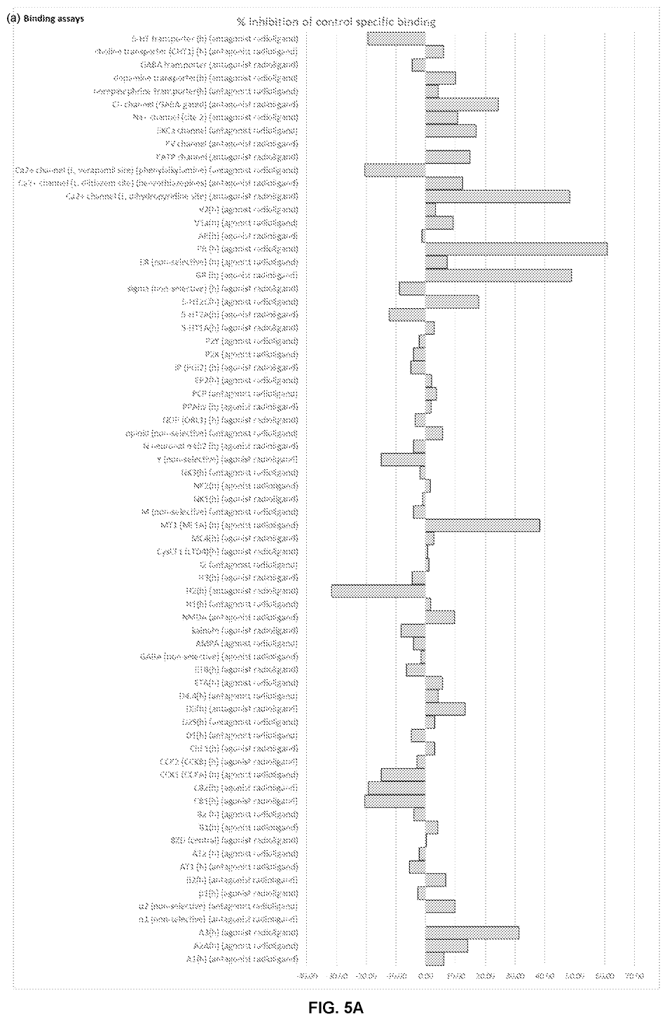

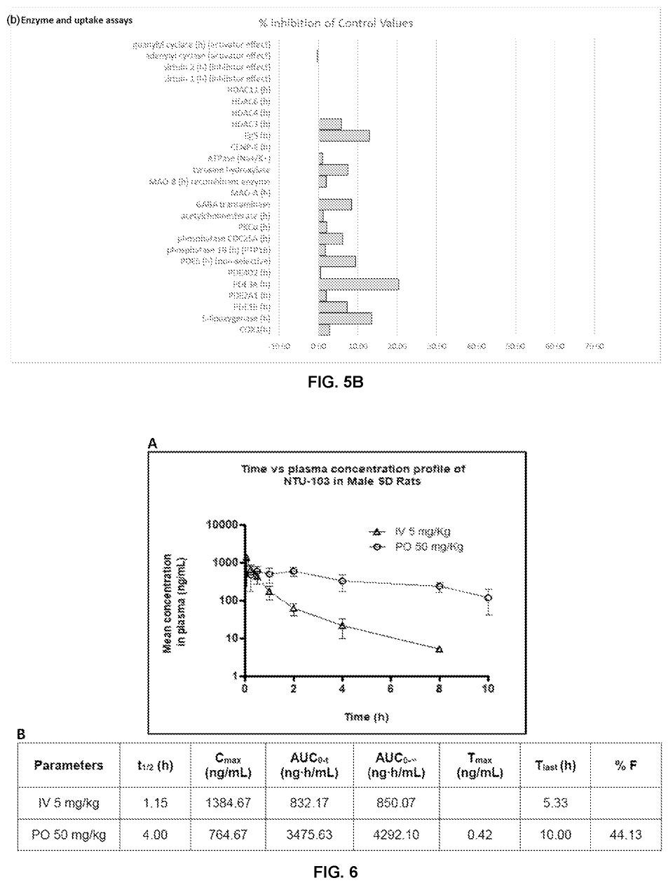

The results are presented in FIG. 5.

### Example 7

The solubility and pharmacokinetics for compound 103 were assessed.

Solubility Test

Calibration standards at 1, 5, 10, 50, 100, 200 and 300 μM were prepared in DMSO from a 20 mM stock. An aliquot of 198 μL of 0.01 M PBS (pH=7.4) was dispensed into duplicate wells of a multiscreen solubility filter plate followed by 2 μL of the test compound solution (20 mM). The plate was covered and shook at 150 rotations per min for 90 min. At the end of 90 min, samples were filtered using MultiScreen HTS vacuum Manifold assembly and the filtrate was collected onto the acceptor plate. An aliquot (150 μL) of the filtrate from the above 96-well acceptor plate was transferred into HPLC vials and analyzed by HPLC-UV. Verapamil, ketoconazole and reserpine were used as high, medium and low soluble reference standards respectively.

Pharmacokinetics Analysis

Male Sprague Dawley (SD) rats (200-240 g) with age between 6-8 weeks were used (n=3, serial sampling). Compound 103 was formulated in DMSO:solutol-ethanol:normal saline (10:10:80; v/v) for intravenous administration (IV) at 5 mg/kg and 5% v/v NMP, 7.5% v/v Solutol HS-15, 50% v/v PEG-400, and 37.5% v/v TPGS (10% w/v in water) for oral administration (PO) at 50 mg/kg. Plasma collection time points for IV and PO administrations were 0.083, 0.25, 0.5, 1, 2, 4, 8, 24 h and 0.25, 0.5, 1, 2, 4, 8, 10, 24 h respectively. Plasma samples were analyzed by LC-MS/MS and the PK parameters were calculated by Phoenix software ver.8.1.

Results and Discussion

Compound 103 was found to be soluble in PBS at pH 7.4 up to 87.4 μM (Table 9).

TABLE 9
Solubility of compound 103 (Group 4c)

| | Solubility (μM) |
|---|---|
| PBS pH 1.5 | 54.6 |
| PBS pH 5.0 | 58.1 |
| PBS pH 7.4 | 87.4 |

The plasma terminal half-life (t_{1/2}) for compound 103 was 1.15 h following a single intravenous injection at 5 mg/kg, and 4 h following a single oral administration at 50 mg/kg in mice. The bioavailability was estimated to be 44.1% in rats (FIG. 6).

### Example 8

The brain penetration ability of compound 103 in mice was evaluated.

Plasma and Brain Distribution Studies

Plasma and brain distribution of compound 103 were determined in male C57BL/6 mice (8-12 weeks old) weighing between 20-35 g, following a single intraperitoneal administration at 25 mg/kg and 50 mg/kg. The formulation vehicle used was 5% v/v NMP, 7.5% v/v Solutol HS-15, 50% v/v PEG-400 and 37.5% v/v TPGS (10% w/v). The dosing volume for intraperitoneal administration was 5 mL/kg. Following blood collection, the animals were sacrificed, and their abdominal vena-cava were cut open and whole body were perfused from heart using 10 mL normal saline. The brain samples were collected from a set of three mice at 0.08, 0.25, 0.5, 1, 2, 4, 8 and 10 h. After isolation, the brain samples were rinsed thrice in ice cold normal saline (for 5-10 s/rinse using ˜10-20 mL normal saline in disposable petri dish for each rinse) and dried on blotting paper. The brain samples were homogenized using ice-cold phosphate buffer saline (pH=7.4). The total homogenate volume was three times the tissue weight. All homogenates were stored below −70° C. until bioanalysis. The concentrations of compound 103 in mouse plasma and brain samples were determined by fit for purpose LC-MS/MS method. Non-Compartmental-Analysis tool of Phoenix WinNonlin® (Version 8.0) was used to assess the pharmacokinetic parameters.

Results and Discussion

Following single intraperitoneal dose administration of compound 103 at 25 mg/kg and 50 mg/kg, peak plasma and brain concentrations were observed at 0.5 h and 0.25 h, respectively, suggesting rapid brain penetration. The terminal elimination half-life was observed at 2.24 h and 1.63 h. Overall, the brain concentrations were higher than plasma with brain-Kp of 1.97 and 4.4 (Table 10).

TABLE 10
Plasma and brain distribution of compound 103 (Group 4c).

| | | Dose | Tₘₐₓ | Cₘₐₓ | AUCₗₐₛₜ | T_{1/2} | MRTₗₐₛₜ | Brain-Kp | Brain-Kp |
|---|---|---|---|---|---|---|---|---|---|
| Matrix | Route | (mg/kg) | (h) | (ng/mL) | (h*ng/mL) | (h) | (h) | (Cₘₐₓ) | (AUCₗₐₛₜ) |
| Plasma | IP | 25 | 0.50 | 4767.57 | 13881.11 | 2.24 | 2.41 | — | — |
| Plasma | IP | 50 | 0.25 | 8541.42 | 20018.27 | 1.63 | 2.02 | — | — |
| Brain^{#} | IP | 25 | 1.00 | 10548.15 | 27363.78 | 2.48 | 2.01 | 2.21 | 1.97 |
| Brain^{#} | IP | 50 | 0.25 | 26768.34 | 88658.12 | 1.06 | 2.09 | 3.13 | 4.4 |

^{#}Brain Cₘₐₓ and AUC are expressed as ng/g and h*ng/g, respectively.

### Example 9

The plasma and liver concentration of compound 103 in mice was evaluated.

Plasma and Liver Studies

Following 14 days of repeated oral dose administration of compound 103 prepared in 5% v/v NMP, 7.5% v/v Solutol HS-15, 50% v/v PEG-400, 37.5% v/v TPGS (10% w/v) at 25 and 50 mg/kg/BID in both male (18.7 to 20.8 g) and female (17.6 to 18.9 g) C57/BL6 mice with an age of 5 to 8 weeks, plasma and liver test item concentrations were quantified using the AB SCIEX LC-MS/MS Triple Quadrapole instrument coupled with Waters UPLC system.

Results and Discussion

The liver to plasma concentration ratio in male and female were 36.03 and 85.59 at 25 mg/kg/BID, and 19.57 and 31.90 at 50 mg/kg/BID (Table 11).

TABLE 11
Plasma and liver concentration compound 103 (Group 4c).

| Dose | | Average Concentration | | Liver to |
|---|---|---|---|---|
| (mg/kg BID) | Gender | Plasma (ng/mL) | Liver (ng/g) | Plasma Ratio |
| 25 | Male | 296.66 | 10148.20 | 36.03 |
| | Female | 100.11 | 8419.33 | 85.59 |
| 50 | Male | 928.51 | 17510.90 | 19.57 |
| | Female | 400.95 | 12075.29 | 31.90 |

### Example 10

The safety profile of compound 103 was assessed by oral administration.

Two-Week Non-GLP Toxicology in Mice

The safety profile of compound 103 was assessed by oral administration, twice daily, at 25 mg/kg and 50 mg/kg in mice for 2 weeks. The formulation used was 5% v/v NMP, 7.5% v/v Solutol HS-15, 50% v/v PEG-400 and 37.5% v/v TPGS (10% w/v). A total of 6 animal groups were used, of which 3 groups were male mice (18.7 to 20.8 g) and 3 groups were female mice (17.6 to 18.9 g), and the mice have an age of 5-8 weeks. Each group contained 6 mice. The animals were analyzed throughout the study, and blood and tissues were analyzed at the end of the study.

Results and Discussion

No abnormal clinical signs were detected, and all animals survived to study termination. The body weight and body weight gain of the mice did not change (Table 12-13) and food consumption was not altered (Table 14).

TABLE 12a
Body weight of male mice.

| | Treatment Day | | | | |
|---|---|---|---|---|---|
| Mean/SD/N | 1 | 4 | 8 | 11 | 14 |

Group: G1 Dose: 0 mg/kg/BID

| | | | | | |
|---|---|---|---|---|---|
| Mean | 18.53 | 18.45 | 19.23 | 19.58 | 20.00 |
| SD | 0.46 | 1.05 | 0.24 | 0.59 | 0.67 |
| N | 6 | 6 | 6 | 6 | 6 |

Group: G2 Dose: 25 mg/kg/BID

| | | | | | |
|---|---|---|---|---|---|
| Mean | 18.30 | 17.65 | 19.12 | 18.95 | 19.27 |
| SD | 0.28 | 0.81 | 0.45 | 1.12 | 0.97 |
| N | 6 | 6 | 6 | 6 | 6 |

Group: G3 Dose: 50 mg/kg/BID

| | | | | | |
|---|---|---|---|---|---|
| Mean | 18.75 | 18.63 | 19.52 | 19.32 | 19.85 |
| SD | 0.43 | 1.07 | 0.41 | 0.58 | 0.69 |
| N | 6 | 6 | 6 | 6 | 6 |

Key: N = Number of animals.

TABLE 12b
Body weight of female mice.

| | Treatment Day | | | | |
|---|---|---|---|---|---|
| Mean/SD/N | 1 | 4 | 8 | 11 | 14 |

Group: G1 Dose: 0 mg/kg/BID

| | | | | | |
|---|---|---|---|---|---|
| Mean | 20.37 | 21.00 | 21.55 | 21.12 | 21.73 |
| SD | 0.49 | 0.69 | 0.53 | 0.69 | 0.52 |
| N | 6 | 6 | 6 | 6 | 6 |

Group: G2 Dose: 25 mg/kg/BID

| | | | | | |
|---|---|---|---|---|---|
| Mean | 20.33 | 21.03 | 21.48 | 21.52 | 21.93 |
| SD | 0.61 | 0.76 | 0.56 | 0.75 | 1.24 |
| N | 6 | 6 | 6 | 6 | 6 |

Group: G3 Dose: 50 mg/kg/BID

| | | | | | |
|---|---|---|---|---|---|
| Mean | 20.47 | 20.37 | 21.13 | 21.20 | 21.73 |
| SD | 0.69 | 0.81 | 1.09 | 0.94 | 0.90 |
| N | 6 | 6 | 6 | 6 | 6 |

Key: N = Number of animals

TABLE 13a
Body weight gain (%) of male mice.

Treatment Day

| | | | | |
|---|---|---|---|---|
| Mean/SD/N | 1 to 4 | 1 to 8 | 1 to 11 | 1 to 14 |

Group: G1 Dose: 0 mg/kg/BID

| | | | | |
|---|---|---|---|---|
| Mean | 3.10 | 5.81 | 3.69 | 6.72 |
| SD | 1.64 | 0.85 | 2.80 | 1.60 |
| N | 6 | 6 | 6 | 6 |

Group: G2 Dose: 25 mg/kg/BID

| | | | | |
|---|---|---|---|---|
| Mean | 3.45 | 5.68 | 5.86 | 7.91 |
| SD | 2.62 | 2.14 | 3.84 | 6.06 |
| N | 6 | 6 | 6 | 6 |

Group: G3 Dose: 50 mg/kg/BID

| | | | | |
|---|---|---|---|---|
| Mean | −0.49*1 | 3.28 | 3.60 | 6.21 |
| SD | 2.01 | 4.51 | 3.68 | 3.43 |
| N | 6 | 6 | 6 | 6 |

Key: N = Number of Animals, *1= Mean value of group significantly decreased from control group at p [symbol] 0.0 [symbol] . [symbol]
indicates data missing or illegible when filed

TABLE 13b
Body weight gain (%) of female mice.

Treatment Day

| | | | | |
|---|---|---|---|---|
| Mean/SD/N | 1 to 4 | 1 to 8 | 1 to 11 | 1 to 14 |

Group: G1 Dose: 0 mg/kg/BID

| | | | | |
|---|---|---|---|---|
| Mean | −0.49 | 3.81 | 5.69 | 7.93 |
| SD | 4.11 | 1.88 | 2.99 | 3.37 |
| N | 6 | 6 | 6 | 6 |

Group: G2 Dose: 25 mg/kg/BID

| | | | | |
|---|---|---|---|---|
| Mean | −3.53 | 4.49 | 3.54 | 5.27 |
| SD | 4.76 | 3.39 | 5.67 | 4.61 |
| N | 6 | 6 | 6 | 6 |

Group: G3 Dose: 50 mg/kg/BID

| | | | | |
|---|---|---|---|---|
| Mean | −0.68 | 4.10 | 3.03 | 5.87 |
| SD | 3.70 | 1.52 | 2.38 | 2.81 |
| N | 6 | 6 | 6 | 6 |

Key: N = Number of Animals.

TABLE 14a
Food consumption by male mice (g/animal).

| | Treatment Day | | | |
|---|---|---|---|---|
| Average Feed Intake/Animal/N | 1 to 4 | 1 to 8 | 1 to 11 | 1 to 14 |

Group: G1 Dose: 0 mg/kg/BID

| | | | | |
|---|---|---|---|---|
| Average Feed Intake/Animal | 11.6 | 14.8 | 8.8 | 10.4 |
| N | 2 | 2 | 2 | 2 |

Group: G2 Dose: 25 mg/kg/BID

| | | | | |
|---|---|---|---|---|
| Average Feed Intake/Animal | 9.9 | 14.8 | 9.5 | 10.4 |
| N | 2 | 2 | 2 | 2 |

Group: G3 Dose: 50 mg/kg/BID

| | | | | |
|---|---|---|---|---|
| Average Feed Intake/Animal | 8.7 | 14.4 | 9.4 | 11.0 |
| N | 2 | 2 | 2 | 2 |

Key: N = Number of Cages.

TABLE 14b
Food consumption by female mice (g/animal).

| | Treatment Day | | | |
|---|---|---|---|---|
| Average Feed Intake/Animal/N | 1 to 4 | 1 to 8 | 1 to 11 | 1 to 14 |

Group: G1 Dose: 0 mg/kg/BID

| | | | | |
|---|---|---|---|---|
| Average Feed Intake/Animal | 9.4 | 13.0 | 9.7 | 9.8 |
| N | 2 | 2 | 2 | 2 |

Group: G2 Dose: 25 mg/kg/BID

| | | | | |
|---|---|---|---|---|
| Average Feed Intake/Animal | 9.4 | 12.8 | 10.3 | 9.5 |
| N | 2 | 2 | 2 | 2 |

Group: G3 Dose: 50 mg/kg/BID

| | | | | |
|---|---|---|---|---|
| Average Feed Intake/Animal | 9.1 | 12.8 | 8.7 | 9.8 |
| N | 2 | 2 | 2 | 2 |

Key: N = Number of Cages.

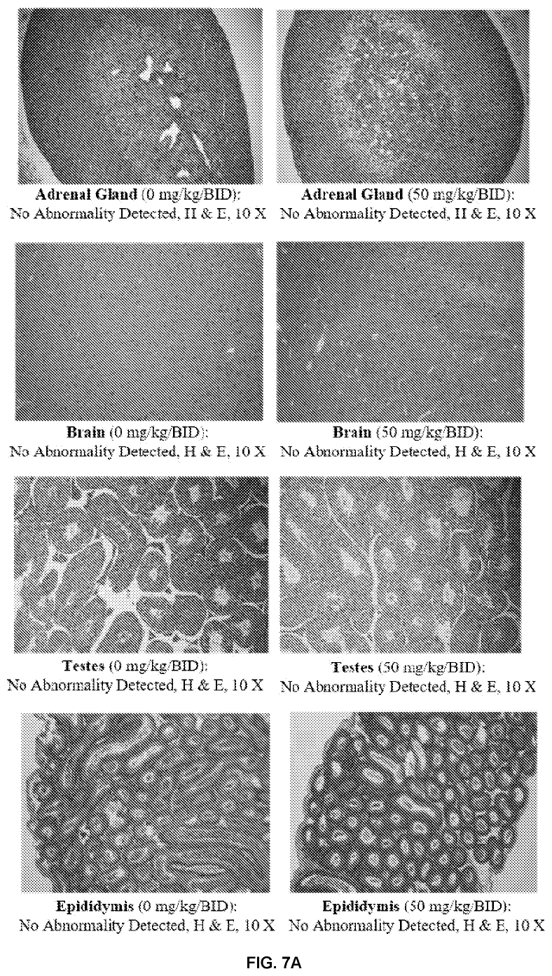

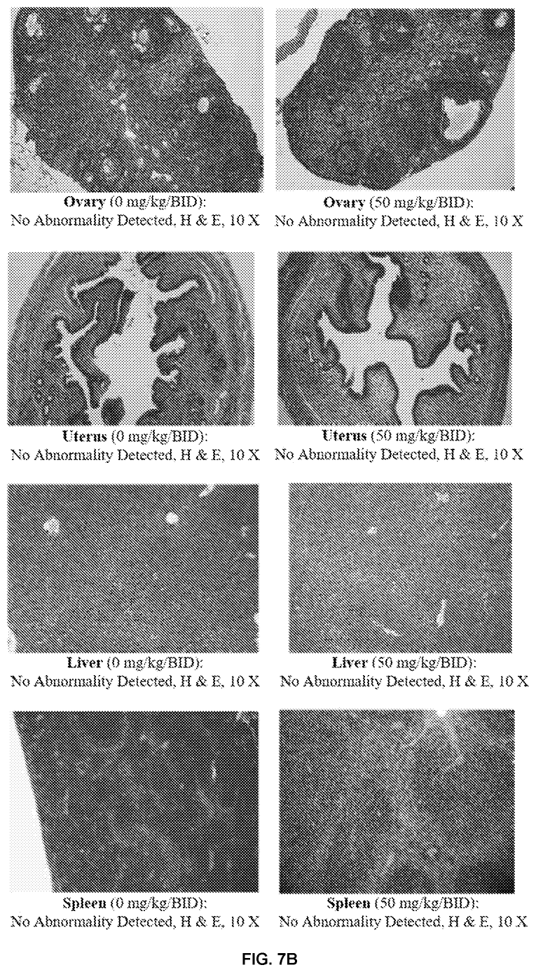

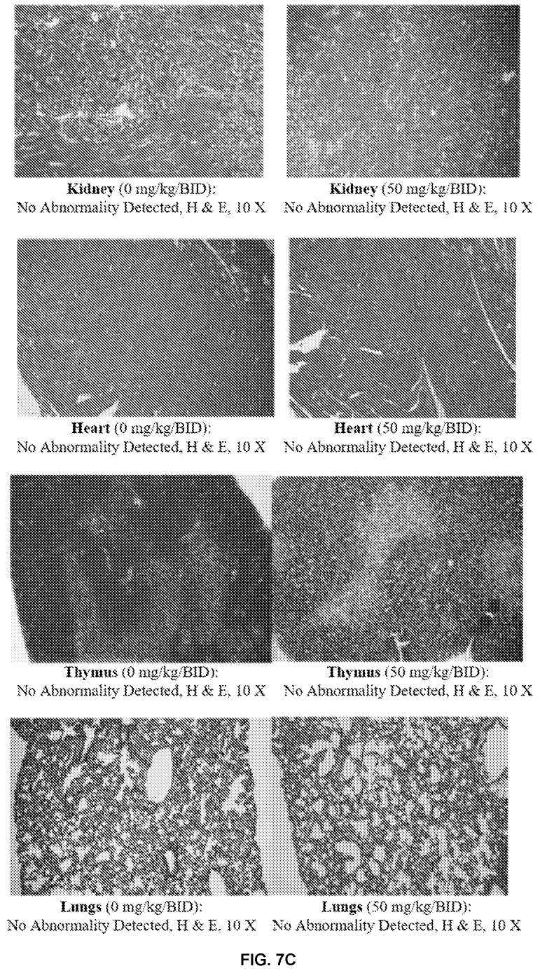

Hematological assessment showed a statistically significant decrease (up to 3.4-fold) in white blood cells (WBC) in 25 mg/kg and 50 mg/kg treated male and female mice. No other toxicologically significant changes were detected (Table 15). In addition, clinical chemistry assessment showed no significant toxicological change in any of the parameters in male and female mice (Table 16). Organ weights were not altered by compound 103 (Table 17), and no obvious gross pathology was noted. Histopathology analysis did not detect any differences with controls (FIG. 7).

TABLE 15a
Hematological parameters of male mice.

| Mean/ | WBC | RBC | HGB | HCT | MCV | MCH | MCHC | PLT |
|---|---|---|---|---|---|---|---|---|
| SD/N | 10³/μl | 10³/μl | g/dL | % | fL | pg | g/dL | 10³/μl |
| Group: G1 | | | | | | | Dose: 0 mg/kg/BID | |
| Mean | 7.73 | 6.73 | 15.97 | 31.43 | 46.63 | 23.70 | 50.77 | 723.00 |
| SD | 0.61 | 0.14 | 0.86 | 0.55 | 0.21 | 1.04 | 2.11 | 189.58 |
| N | 3 | 3 | 3 | 3 | 3 | 3 | 3 | 3 |
| Group: G2 | | | | | | | Dose: 25 mg/kg/BID | |
| Mean | 3.70*↓ | 6.59 | 15.37 | 31.43 | 47.70 | 23.27 | 48.87 | 591.33 |
| SD | 0.52 | 0.19 | 1.61 | 0.81 | 0.82 | 1.78 | 4.54 | 121.25 |
| N | 3 | 3 | 3 | 3 | 3 | 3 | 3 | 3 |
| Group: G3 | | | | | | | Dose: 50 mg/kg/BID | |
| Mean | 2.27*↓ | 6.34 | 13.73 | 30.70 | 48.40*↑ | 21.70 | 44.77 | 661.00 |
| SD | 0.58 | 0.31 | 0.75 | 1.73 | 0.78 | 0.35 | 0.06 | 15.62 |
| N | 3 | 3 | 3 | 3 | 3 | 3 | 3 | 3 |

| Mean/SD/N | Neutrophils | Lymphocytes | Monosytes | Eosinophils | Basophils |
|---|---|---|---|---|---|
| Group: G1 | | | | Dose: 0 mg/kg/BID | |
| Mean | 15.00 | 82.67 | 2.00 | 0.33 | 0.00 |
| SD | 1.73 | 2.08 | 1.00 | 0.58 | 0.00 |
| N | 3 | 3 | 3 | 3 | 3 |
| Group: G2 | | | | Dose: 25 mg/kg/BID | |
| Mean | 15.67 | 82.33 | 1.33 | 0.67 | 0.00 |
| SD | 5.69 | 5.69 | 0.58 | 0.58 | 0.00 |
| N | 3 | 3 | 3 | 3 | 3 |
| Group: G3 | | | | Dose: 50 mg/kg/BID | |
| Mean | 20.00 | 78.00 | 2.00 | 0.00 | 0.00 |
| SD | 7.00 | 7.00 | 0.00 | 0.00 | 0.00 |
| N | 3 | 3 | 3 | 3 | 3 |

| Mean/SD/N | Reticuloxyte Count | |
|---|---|---|
| Group: G1 | | Dose: 0 mg/kg/BID |
| Mean | 2.77 | |
| SD | 0.50 | |
| N | 3 | |
| Group: G2 | | Dose: 25 mg/kg/BID |
| Mean | 2.90 | |
| SD | 0.50 | |
| N | 3 | |
| Group: G3 | | Dose: 50 mg/kg/BID |
| Mean | 2.67 | |
| SD | 0.32 | |
| N | 3 | |

[symbol]

[symbol]

indicates data missing or illegible when filed

TABLE 15b
Hematological parameters of female mice.

| Mean/ | WBC | RBC | HGB | HCT | MCV | MCH | MCHC | PLT |
|---|---|---|---|---|---|---|---|---|
| SD/N | 10³/μl | 10³/μl | g/dL | % | fL | pg | g/dL | 10³/μl |
| Group: G1 | | | | | | | Dose: 0 mg/kg/BID | |
| Mean | 6.77 | 6.21 | 14.67 | 28.13 | 45.36 | 23.57 | 52.07 | 704.00 |
| SD | 1.05 | 0.03 | 0.99 | 0.25 | 0.26 | 1.68 | 3.84 | 10.58 |
| N | 3 | 3 | 3 | 3 | 3 | 3 | 3 | 3 |
| Group: G2 | | | | | | | Dose: 25 mg/kg/BID | |
| Mean | 3.73*↓ | 6.15 | 12.97 | 28.87 | 46.97*↑ | 21.10*↓ | 44.93*↓ | 616.67 |
| SD | 0.47 | 0.35 | 0.98 | 2.12 | 0.81 | 0.40 | 0.35 | 99.57 |
| N | 3 | 3 | 3 | 3 | 3 | 3 | 3 | 3 |
| Group: G3 | | | | | | | Dose: 50 mg/kg/BID | |
| Mean | 2.17*↓ | 6.14 | 13.67 | 28.73 | 46.83*↑ | 21.23 | 45.37*↓ | 733.00 |
| SD | 0.45 | 0.02 | 0.23 | 0.42 | 0.65 | 0.68 | 0.90 | 49.51 |
| N | 3 | 3 | 3 | 3 | 3 | 3 | 3 | 3 |
| Mean/ | | | | | | | | |

| SD/N | Neutrophils | Lympthocytes | Monocytes | Eosinophils | Basophils |
|---|---|---|---|---|---|
| Group: G1 | | | | Dose: 0 mg/kg/BID | |
| Mean | 11.00 | 86.67 | 2.00 | 0.33 | 0.00 |
| SD | 1.00 | 1.15 | 1.00 | 0.38 | 0.00 |
| N | 3 | 3 | 3 | 3 | 3 |
| Group: G2 | | | | Dose: 25 mg/kg/BID | |
| Mean | 17.00 | 82.00 | 1.00 | 0.00 | 0.00 |
| SD | 9.85 | 8.89 | 1.00 | 0.00 | 0.00 |
| N | 3 | 3 | 3 | 3 | 3 |
| Group: G3 | | | | Dose: 50 mg/kg/BID | |
| Mean | 14.67 | 84.00 | 1.33 | 0.00 | 0.00 |
| SD | 4.16 | 3.61 | 0.58 | 0.00 | 0.00 |
| N | 3 | 3 | 3 | 3 | 3 |

| Mean/SD/N | Reticuloxyte Count | |
|---|---|---|
| Group: G1 | | Dose: 0 mg/kg/BID |
| Mean | 2.83 | |
| SD | 0.23 | |
| N | 3 | |
| Group: G2 | | Dose: 25 mg/kg/BID |
| Mean | 2.63 | |
| SD | 0.25 | |
| N | 3 | |
| Group: G3 | | Dose: 50 mg/kg/BID |
| Mean | 2.67 | |
| SD | 0.06 | |
| N | 3 | |

[symbol]

[symbol]

indicates data missing or illegible when filed

TABLE 16a
Clinical Chemistry assessment of male mice.

| Mean/SD/N | ALT | ALB | ALP | AST | BUN | CREA |
|---|---|---|---|---|---|---|
| Group: G1 | | | | | Dose: 0 mg/kg/BID | |
| Mean | 64.67 | 2.36 | 199.58 | 108.35 | 18.92 | 0.16 |
| SD | 16.91 | 0.14 | 27.41 | 26.01 | 1.15 | 0.01 |
| N | 3 | 3 | 3 | 3 | 3 | 3 |
| Group: G2 | | | | | Dose: 25 mg/kg/BID | |
| Mean | 42.36 | 2.66 | 207.96 | 79.38 | 19.73 | 0.14 |
| SD | 7.94 | 0.0.9 | 17.43 | 29.11 | 5.12 | 0.02 |
| N | 3 | 3 | 3 | 3 | 3 | 3 |
| Group: G3 | | | | | Dose: 50 mg/kg/BID | |
| Mean | 59.72 | 2.43 | 180.63 | 88.76 | 22.97 | 0.12*↓ |
| SD | 33.63 | 0.23 | 9.45 | 21.99 | 1.64 | 0.02 |
| N | 3 | 3 | 3 | 3 | 3 | 3 |
| | GLU | LDLC | TBIL | TP | TGL | Urea |
| Mean/SD/N | mg/dL | mg/dL | mg/dL | g/dL | mg/dL | mg/dL |
| Group: G1 | | | | | Dose: 0 mg/kg/BID | |
| Mean | 156.56 | 8.78 | 0.27 | 3.54 | 68.48 | 40.53 |
| SD | 16.24 | 0.56 | 0.05 | 0.06 | 23.32 | 2.46 |
| N | 3 | 3 | 3 | 3 | 3 | 3 |
| Group: G2 | | | | | Dose: 25 mg/kg/BID | |
| Mean | 157.88 | 10.02 | 0.09*↓ | 4.13*↑ | 73.46 | 42.29 |
| SD | 29.96 | 0.59 | 0.01 | 0.09 | 40.19 | 10.97 |
| N | 3 | 3 | 3 | 3 | 3 | 3 |
| Group: G3 | | | | | Dose: 50 mg/kg/BID) | |
| Mean | 152.77 | 9.84 | 0.08*↓ | 4.19*↑ | 84.42 | 47.30 |
| SD | 17.30 | 1.47 | 0.02 | 0.17 | 31.58 | 3.50 |
| N | 3 | 3 | 3 | 3 | 3 | 3 |

[symbol]

[symbol]

indicates data missing or illegible when filed

TABLE 16b
Clinical Chemistry assessment of female mice.

| | ALT | ALB | ALP | AST | BUN | CREA |
|---|---|---|---|---|---|---|
| Mean/SD/N | U/L | g/dL | U/L | U/L | mg/dL | mg/dL |
| Group: G1 | | | | | Dose: 0 mg/kg/BID | |
| Mean | 65.32 | 2.89 | 316.87 | 81.22 | 23.78 | 0.13 |
| SD | 36.82 | 0.15 | 43.16 | 22.92 | 1.35 | 0.01 |
| N | 3 | 3 | 3 | 3 | 3 | 3 |
| Group: G2 | | | | | Dose: 25 mg/kg/BID | |
| Mean | 40.03 | 2.60*↓ | 303 38 | 84.12 | 19.24 | 0.13 |
| SD | 5.10 | 0.02 | 92.27 | 43.48 | 4.05 | 0.01 |
| N | 3 | 3 | 3 | 3 | 3 | 3 |
| Group: G3 | | | | | Dose: 50 mg/kg/BID | |
| Mean | 55.57 | 2.55*↓ | 254.38 | 92.78 | 22.71 | 0.12 |
| SD | 2.12 | 0.11 | 33.93 | 43.62 | 1.73 | 0.01 |
| N | 3 | 3 | 3 | 3 | 3 | 3 |
| | GLU | LDLC | TBIL | TP | TGL | Urea |
| Mean/SD/N | mg/dL | mg/dL | mg/dL | g/dL | mg/dL | mg/dL |
| Group: G1 | | | | | Dose: 0 mg/kg/BID | |
| Mean | 165.74 | 11.23 | 0.14 | 4.24 | 70.66 | 50.94 |
| SD | 11.35 | 0.73 | 0.06 | 0.27 | 3.14 | 2.89 |
| N | 3 | 3 | 3 | 3 | 3 | 3 |
| Group: G2 | | | | | Dose: 25 mg/kg/BID | |
| Mean | 153.02 | 9.31 | 0.12 | 4.12 | 48.73 | 41.23 |
| SD | 18.85 | 2.90 | 0.02 | 0.09 | 6.18 | 8.68 |
| N | 3 | 3 | 3 | 3 | 3 | 3 |
| Group: G3 | | | | | Dose: 50 mg/kg/BID | |
| Mean | 165.87 | 8.23 | 0.14 | 3.89 | 59.97 | 48.65 |
| SD | 4.15 | 0.85 | 0.07 | 0.29 | 17.13 | 3.71 |
| N | 3 | 3 | 3 | 3 | 3 | 3 |

[symbol]

[symbol]

indicates data missing or illegible when filed

TABLE 17a
Organ weights relative to body weight (%) of male mice.

| Mean/SD/N | Adrenals | Testes | Epididy [symbol] | Liver | Spleen | Kidneys | Heart | Thy [symbol] | Brain |
|---|---|---|---|---|---|---|---|---|---|
| Group: G1 | | | | | | | | Dose: 0 mg/kg/BID | |
| Mean | 0.024 | 0.750 | 0.237 | 7.450 | 0.499 | 1.581 | 0.477 | 0.133 | 2.035 |
| SD | 0.002 | 0.052 | 0.044 | 0.474 | 0.101 | 0.182 | 0.035 | 0.011 | 0.138 |
| N | 3 | 3 | 3 | 3 | 3 | 3 | 3 | 3 | 3 |
| Group: G2 | | | | | | | | Dose: 25 mg/kg/BID | |
| Mean | 0.024 | 0.582 | 0.333 | 7.093 | 0.351 | 1.490 | 0.500 | 0.136 | 2.065 |
| SD | 0.009 | 0.135 | 0.030 | 0.720 | 0.038 | 0.122 | 0.022 | 0.009 | 0.114 |
| N | 3 | 3 | 3 | 3 | 3 | 3 | 3 | 3 | 3 |
| Group: G3 | | | | | | | | Dose: 50 mg/kg/BID | |
| Mean | 0.030 | 0.773 | 0.298 | 7.014 | 0.461 | 1.467 | 0.541 | 0.116 | 2.017 |
| SD | 0.009 | 0.157 | [symbol] | 0.338 | [symbol] | 0.051 | 0.045 | 0.031 | 0.082 |
| N | 3 | 3 | 3 | 3 | | 3 | 3 | 3 | 3 |

[symbol]

[symbol]

indicates data missing or illegible when filed

TABLE 17b
Organ weights relative to body weight (%) of female mice.

| Mean/SD/N | Adrenals | Ovaries | U [symbol] | Liver | Spleen | Kidneys | Heart | Thymus | Brain |
|---|---|---|---|---|---|---|---|---|---|
| Group: G1 | | | | | | | | Dose: 0 mg/kg/BID | |
| Mean | 0.033 | 0.056 | 0.388 | 0.237 | 0.369 | 1.350 | 0.549 | 0.257 | 2.174 |
| SD | 0.012 | 0.013 | 0.075 | 0.025 | 0.034 | 0.033 | 0.050 | 0.036 | 0.133 |
| N | 3 | 3 | 3 | 3 | 3 | 3 | 3 | 3 | 3 |
| Group: G2 | | | | | | | | Dose: 25 mg/kg ROD | |
| Mean | 0.038 | 0.077 | 0.321 | 6.436 | 0.672*↑ | 1.379 | 0.990 | 0.200 | 2.217 |
| SD | 0.005 | 0.014 | 0.267 | 0.489 | 0.068 | 0.048 | 0.721 | 0.025 | 0.083 |
| N | 3 | 3 | 3 | 3 | 3 | 3 | 3 | 3 | 3 |
| Group: G3 | | | | | | | | Dose: 50 mg/kg:BID | |
| Mean | 0.035 | 0.066 | 0.277 | 7.356 | 0.490 | 1.375 | 0.532 | 0.220 | 2.175 |
| SD | 0.004 | 0.018 | 0.054 | 1.026 | 0.087 | 0.109 | 0.011 | 0.043 | 0.092 |
| N | 3 | 3 | 3 | 3 | 3 | 3 | 3 | 3 | 3 |

[symbol]

[symbol]

indicates data missing or illegible when filed

Hence it can be concluded that when compound 103 was administered twice daily for 14 consecutive days by oral route to C57BL/6 mice, the tolerable dose was >50 mg/kg/BID.

### Example 11

Of the many potential clinical indications that have been experimentally validated based on K_{Ca}3.1 gene knockout or selective K_{Ca}3.1 blockade, as a proof-of-concept study we have assessed compound 103 in an animal model for one clinical indication, namely stroke. Genetic knockout or pharmacological blockade of K_{Ca}3.1 in mouse and rat models of ischemic/reperfusion stroke reduced infarct size, microglia-mediated neuroinflammation and astrogliosis, and improved neurological scores (Y. J. Chen et al., J. Cereb. Blood Flow Metab. 2011, 31, 2363-2374; M. Yi et al., J. Neuroinflammation 2017, 14, 203; M. S. V. Elkind et al., Neurology 2020, 95, e1091-e1104; and Z. Yu et al., Front. Cell. Neurosci. 2017, 11, 319). Since compound 103 has excellent brain penetration, a proof-of-concept study of compound 103 in a rodent model of ischemic stroke was carried out to determine if compound 103 is effective in treating stroke by suppressing neuro-inflammation-mediated secondary brain damage following acute ischemia-reperfusion stroke.

MCAO Ischemia-Reperfusion Model

As a positive control, Edaravone, a clinically-approved first-in-class medication used to help patients recover following a stroke, was used.

Briefly, SD male rats weighing 240-270 g were used for the MCAO study with 7 days of reperfusion. Focal cerebral schemia was induced by occlusion of the right middle cerebral artery (MCA). A median incision in the neck was made to expose the right common carotid artery, internal carotid artery and external carotid artery. A slipknot on the common carotid artery, a dead knot on the proximal side of the external carotid artery, and a slipknot on the external carotid artery near the common carotid bifurcation were tied with a silk thread. A small opening between the two knots of the external carotid artery was cut to insert a thread plug into the carotid artery and advance inwardly into the middle cerebral artery. The plug was kept in place for 60 m and then withdrawn and removed from the blood vessel to restore blood supply. After 12 h reperfusion, the mice received compound 103 at 2, 5.10 mg/kg or vehicle through intraperitoneal injection every 12 h, for 7 days. Edaravone at 5 mg/kg was given intraperitoneally daily for 7 days as a reference control. Assessment of infarct area was done by 2,3,5-triphenyltetrazolium chloride (TTC) staining of the whole brain sections (2 mm thick) starting from the frontal pole and scanned images were analyzed with ImageJ.

Neurological Behavioral Evaluation

The animals were individually assessed based on the scoring method described in Table 18.

TABLE 18
Neurological behavioural scoring method.

| | Test | Score |
|---|---|---|
| General | Irritability/Long Hair | 1 |
| behavior | Still/staring | 1 |
| | Seizures/muscle spasms/tremors | 1 |
| | Forelimb flexion | 1 |
| | Hind limb flexion | 1 |
| | The head deviates from the vertical axis >10° | 1 |
| | within 30 s | |
| movement | Can't walk straight | 1 |
| | A little bit of a circle | 2 |
| | Turn around (>50%) | 3 |
| | Fall down | 4 |
| Senses | Corneal reflex disappeared | 1 |
| | Loss of auricle reflex | 1 |
| | Startle reflex disappeared | 1 |
| Proprioceptive | Hold on to the edge of the balance beam | 1 |
| officer (on | Holding the balance beam tightly, one limb | 2 |
| balance beam) | slipped off | |
| | Hold the balance beam tightly, and the limbs | 3 |
| | fall or rotate from the balance beam (>60 s) | |
| | Trying to balance on the balance beam but | 4 |
| | falling (5 s) | |
| | Unable to balance and fall directly | 5 |
| Total score | | 18 |

| | |
|---|---|
| Remarks | 1-6 points for mild injury, 7-12 points for moderate |
| | injury, 13-18 points for severe injury |

Results and Discussion

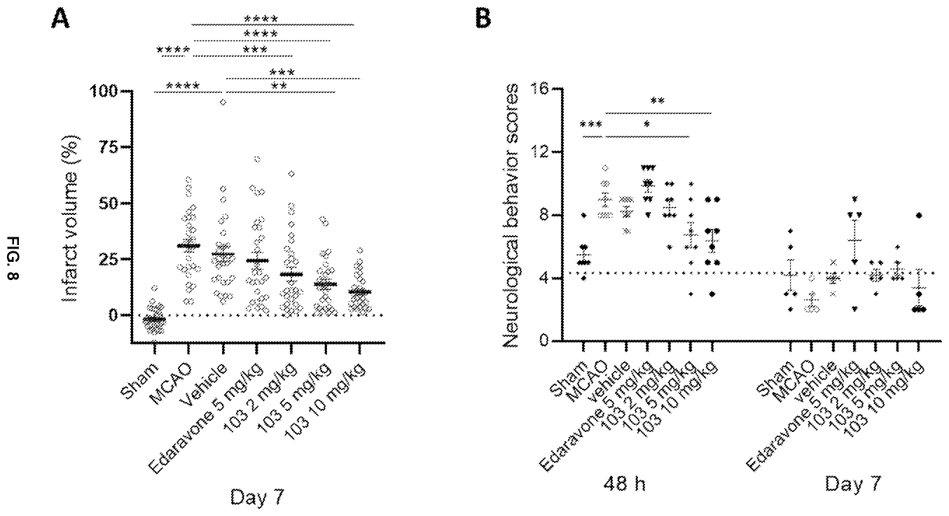

Compound 103 administered twice daily by intraperitoneal injection effectively reduced infarct volume, by nearly 50% at 10 mg/kg, and was more effective than Edaravone (5 mg/kg), the positive control (FIG. 8A). Neurological behavioral evaluation using an 18-point system suggested that compound 103 improves neurological behavior scores more effectively than Edaravone (FIG. 8B).

### Example 12

Immunohistochemistry Analysis on Microglia and Leukocytes Markers

The animals were anaesthetized with isoflurane (RWD Life Science), perfused and fixed with 4% PFA (Sinopharm Chemical Reagent). The brain tissues were further soaked in 4% PFA for 4 h at 4° C. overnight and then treated with a gradient of sucrose solutions (20-30%, Sinopharm Chemical Reagent) before being embedded with OCT (Leica) and frozen at −80° C. For immunofluorescence staining, the brain slices from primary somatosensory cortex (S1), primary motor cortex (M1), field CA1 of hippocampus (CA1) and caudate putamen (striatum, CPu) were heated for 30 min and then washed with 0.01 M PBS (pH=7.2-7.4, 5 min×3). After washing, the sections were blocked with 0.3% TritonX-100 (Solarbio) in 10% sheep serum (Solarbio) at RT for 1 h. Following removal of the blocking solution, the brain sections were incubated with primary antibodies Iba1 (Proteintech, Cat.10904-1-AP) and CD11b (Thermo Fisher, Cat. MA5-17857) overnight at 4° C. Following 3 washes with 0.01 M PBS (5 min each), the sections were incubated with Backlight plus secondary antibody (Abcam, Cat. ab150113; Cat.ab150080) at RT for 2 h. After 3 washes, the nuclei were stained with 4′, 6-diamidino-2-phenylindole (DAPI, Beyotime Biotech, Cat. C1006). Images of whole brain sections were acquired by confocal laser scanning microscope (CLSM, Leica STELLARIS 5, Germany). We used two markers to visualize microglia, Iba1 (17-kDa EF hand protein that is expressed in microglia and is upregulated during the activation of microglia) and CD11b (α-chain of integrin receptor CD11b/CD18 (also known as α_{Mβ2}, Mac-1, and CR3), is highly expressed on the surface of innate immune cells including microglia). The number of Iba1⁺CD11b⁺ double positive microglia were analyzed by Image J V1.8.0. Microglia/macrophage activation is presented as the number of Iba1 and CD11b colocalized cells per section.

Results and Discussion

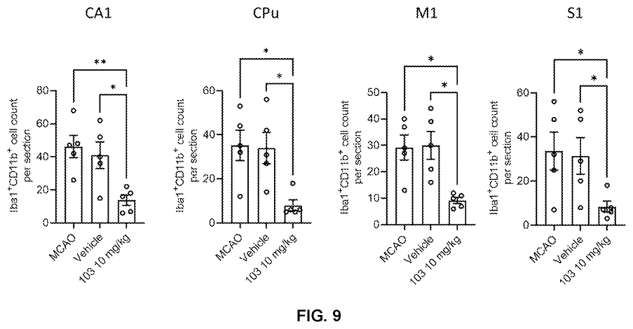

Treatment with compound 103 significantly reduced the number of Iba1⁺CD11b⁺ double positive microglia on day 7 (FIG. 9) in brain areas of CA1, CPu, M1 and S1, compared to MCAO or vehicle groups. Compound 103, when administered starting 12 h after reperfusion in rats, reduced microglia activation on CD11b microglia.

In summary, compared to other analogues in our series, compound 103 is less plasma protein bound, has lower c Log P values, is more soluble, accumulates at concentration higher in the brain than plasma after single dose administration and does not result in abnormal clinical signs and mortality after 14-day repeated daily doses at 25 and 50 mg/kg/BID. Further, in the exploratory proof-of-concept study, compound 103 was effective in reducing infarct volume, microglia activation and improving neurological and behavior scores in a rat model of ischemia/reperfusion stroke.

1. A compound of formula I: 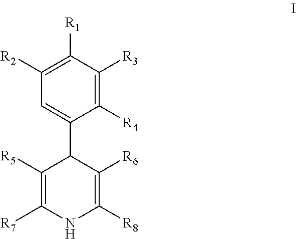
where:
R₁ is selected from H, halo, CF₃, CN or NO₂;
R₂ and R₃ are independently selected from H, halo, CH₃, CF₃, CN or NO₂;
R₄ is selected from H, halo, CN or CF₃;
R₅ and R₆ are independently selected from R₉ₐC(O)O—, R_{9b}OC(O)—, R_{9c}C(O)NR_{d}—, R₉ₑR_{9f}NC(O)—, or an alkyl ketone having from 1 to 10 carbon atoms, which carbon atoms are branched or unbranched and are unsubstituted or substituted by one of more substituents selected from halo, and NO₂;
R₇ and R₈ are independently selected from H, NR₁₀ₐR_{10b}, OR_{10c} or C₁ to C₃ alkyl which is unsubstituted or substituted by one or more substituents selected from halo, or
one of the pair of R₅ and R₇ or R₆ and R₈, together with the carbon atoms that they are attached to, form a 4- to 14-membered ring system that is carbocyclic or heterocyclic and which is unsubstituted or substituted by one or more substituents selected from halo, ═O, —OC(O)R_{10d}, —(O)COR₁₀ₑ, and C₁ to C₆ alkyl;
R₉ₐ to R_{9f} and R₁₀ₐ to R₁₀ₑ are independently selected from H and C₁ to C₆ alkyl which is unsubstituted or substituted by one or more substituents selected from halo, or pharmaceutically acceptable salts and/or solvates thereof.

2. The compound according to claim 1, wherein one or more of the following apply:
(a) R₁ is selected from H, F, Cl, Br, CF₃ or NO₂;
(b) R₂ and R₃ are independently selected from H, F, Cl, Br, CH₃, CF₃ or NO₂;
(c) R₄ is selected from H, F, Cl, Br, or CF₃; and
(d) R₅ and R₆ are independently selected from R₉ₐC(O)O—, R_{9b}OC(O)—, or an alkyl ketone having from 1 to 10 carbon atoms, which carbon atoms are branched or unbranched and are unsubstituted or substituted by one of more substituents selected from Cl, F, and NO₂.

3. (canceled)

4. (canceled)

5. (canceled)

6. The compound according claim 1, wherein R₇ and R₈ are independently selected from H or C₁ to C₃ alkyl which is unsubstituted or substituted by one or more substituents selected from F and Cl, or
one of the pair of R₅ and R₇ or R₆ and R₈, together with the carbon atoms that they are attached to, form a 4- to 10-membered ring system that is carbocyclic or heterocyclic and which is unsubstituted or substituted by one or more substituents selected from F, Cl, ═O, and C₁ to C₆ alkyl.

7. The compound according to claim 1, wherein R₉ₐ to R_{9f} and R₁₀ₐ to R₁₀ₑ are independently selected from H and C₁ to C₃ alkyl which is unsubstituted or substituted by one or more substituents selected from F and Cl.

8. The compound according to claim 1, wherein:
R₁ is selected from H, F, Cl, or CF₃;
R₂ and R₃ are independently selected from H, F, Cl, or CF₃;
R₄ is selected from H, F, Cl, or CF₃;
R₅ and R₆ are independently selected from R_{9b}OC(O)—, an alkyl ketone having from 1 to 3 carbon atoms, which carbon atoms are unsubstituted or substituted by one of more substituents selected from Cl and F;
R₇ and R₈ are independently selected from H, or methyl which is unsubstituted or substituted by one or more substituents selected from F and Cl.

9. The compound according to claim 1, wherein:
R₁ is selected from H, F, or Cl; and/or
R₂ is selected from CF₃ or, more particularly, H or F; and/or
R₃ is selected from H or CF₃; and/or
R₄ is H; and/or
R₅ and R₆ are independently selected from CH₃OC(O)— or propan-2-onyl (e.g. R₅ and R₆ are both CH₃OC(O)—); and/or
R₇ and R₈ are independently selected from H and CH₃.

10. The compound according to claim 1, wherein at least one of R₇ and R₈ is H.

11. The compound according to claim 1, wherein both of R₇ and R₈ is H.

12. The compound according to claim 1, wherein R₇ is H and R₈ is CH₃.

13. The compound according to claim 1, wherein R₇ and R₈ are both CH₃.

14. The compound according to claim 1, wherein R₇ is CH₃ and R₈ is H.

15. The compound according to claim 1 selected from the list, 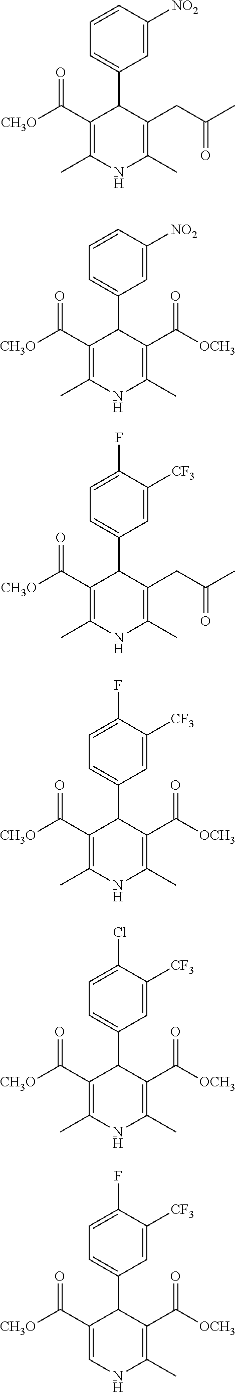 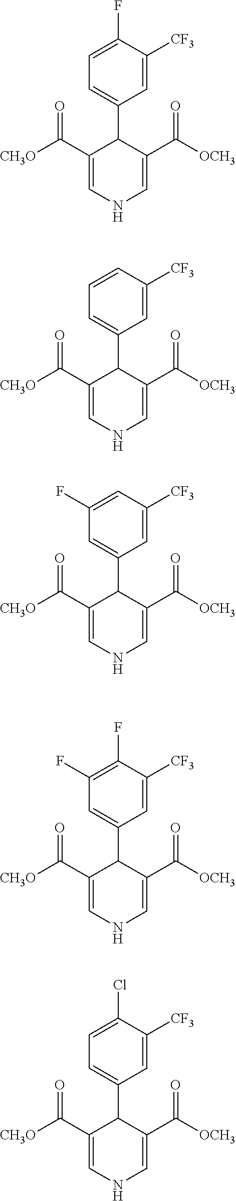
or salts and solvates thereof.

16. The compound according to claim 1 selected from the list, 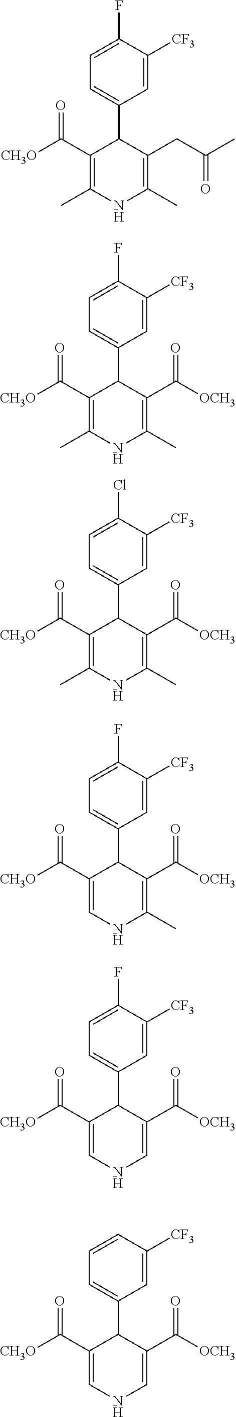 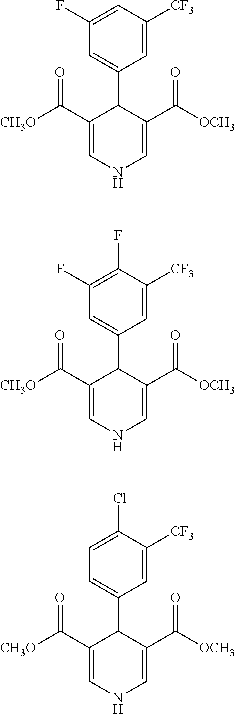
or salts and solvates thereof.

17. The compound according to claim 1 selected from the list, 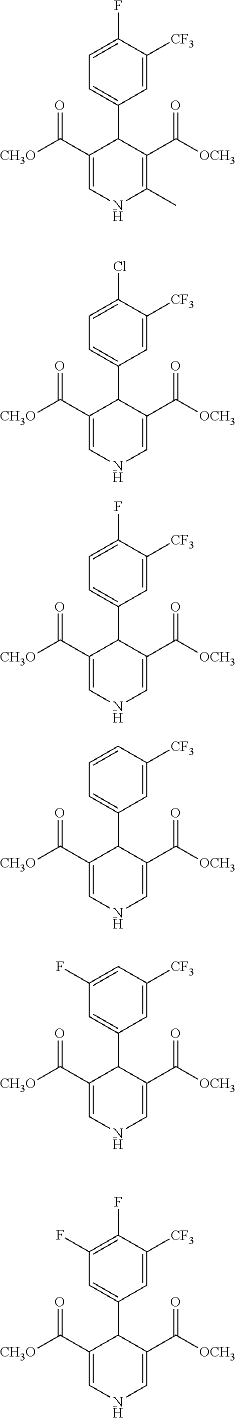
or salts and solvates thereof.

18. The compound according to claim 1 selected from the list, 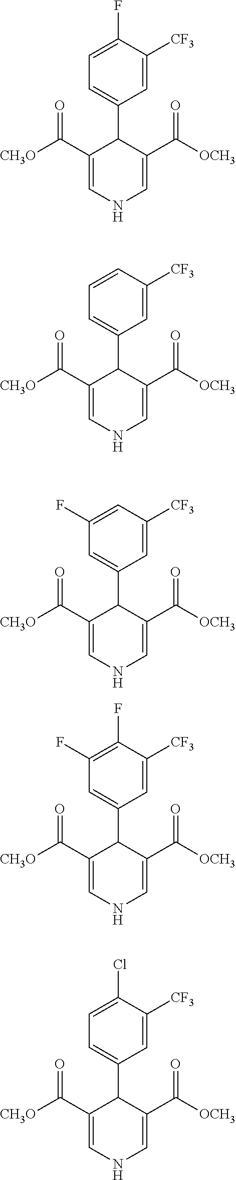
or salts and solvates thereof.

19. The compound according to claim 1 wherein the compound is 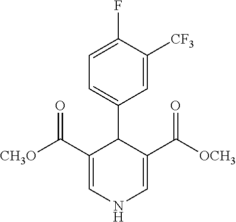
or salts and solvates thereof.

20. A pharmaceutical composition comprising a compound of formula I, or salts and solvates thereof, as described in claim 1 in combination with one or more of a pharmaceutically acceptable carrier, adjuvant, or vehicle.

21. (canceled)

22. (canceled)

23. (canceled)

24. A method of treatment or prevention of a disease condition that is associated with increased K_{Ca}3.1 or altered activity, wherein the method comprises administering an effective amount of a compound of formula, or salts and solvates thereof, as described in claim 1 to a subject in need thereof.

25. The method of claim 24, wherein the disease condition that is associated with increased K_{Ca}3.1 or altered activity is selected from one or more of inflammatory bowel diseases (IBD), fibrotic diseases (lung, liver, renal, cardiac, conjunctival, corneal), non-alcoholic fatty liver disease (NAFLD), nonalcoholic steatohepatitis (NASH), gliomas (glioblastoma), lung cancer, pancreatic cancer, hepatocellular carcinoma, ovarian cancer, colorectal cancer, cystic fibrosis, diabetic renal disease, glomerulonephritis, bone resorption, inflammatory arthritis, multiple sclerosis, atherosclerosis, restenosis following angioplasty, in-stent neo-atherosclerosis, stroke, traumatic brain injury, Alzheimer's disease, hereditary xerocytosis, sickle cell anemia, leukaemia, asthma, allergic rhinitis, microglial activation, nitric oxide-dependent neurodegeneration, neuro-oncological diseases and orphan red blood cell disorders.

26. (canceled)

27. The method of claim 25, wherein the disease condition is stroke.
